(19) United States
(12) Patent Application Publication
(10) Pub. No.: US 2010/0317577 A1
(43) Pub. Date: Dec. 16, 2010

(54) Methods and Compositions for Inhibiting Cell Death or Enhacing Cell Proliferation

(75) Inventors: Richard J Youle, Chevy Chase, MD (US); Antonella Antignani, North Bethesda, MD (US)

Correspondence Address: OTT-NIH;C/O EDWARDS ANGELL PALMER & DODGE LLP, PO BOX 55874, BOSTON, MA, 02205, US

(73) Assignee: Department of Health and Human Services, Rockville, MD (US)

(21) Appl. No.: 11/991,692

(22) PCT Filed: Sep. 8, 2006
(86) PCT No.: PCT/US2006/035070

§ 371 Date: Aug. 26, 2010

Related U.S. Application Data
(60) Provisional application No. 60/715,722, filed on Sep. 9, 2005.

(51) Int. Cl. A61K 38/18; C07K 14/535; C07K 7/06; C07K 7/08; C07H 21/04; C12N 15/63; C12N 5/071; G01N 33/53; A61P 25/28

(52) U.S. Cl. 514/7.6; 530/350; 530/324; 530/327; 530/326; 536/23.5; 435/320.1; 435/325; 435/375; 435/7.21

(57) ABSTRACT

The present invention provides compositions and methods that enhance cell survival. Such compositions feature chimeric polypeptides that include at least a GM-CSF receptor ligand and an anti-apoptotic moiety (e.g., a Bcl-2 protein family member). In one embodiment, the chimeric polypeptide is a GM-CSF-Bcl-xL chimeric polypeptide. The invention further includes methods of using chimeric polypeptides to enhance cell survival or inhibit cell death in a cell at risk of cell death.

### CROSS-REFERENCE TO RELATED APPLICATION

This application claims the benefit of U.S. Provisional Application Nos. 60/715,722, which was filed on Sep. 9, 2006, the entire contents of which are incorporated herein by reference.

### STATEMENT OF RIGHTS TO INVENTIONS MADE UNDER FEDERALLY SPONSORED RESEARCH

This work was supported by a National Institute of Neurological Disorders and Stroke, National Institutes of Health, Bethesda, Md. The government may have certain rights in the invention.

### BACKGROUND OF THE INVENTION

Programmed cell death, also termed “apoptosis,” is common during animal development. Apoptosis is subject to positive and negative regulation. Where this regulation fails, disease results. Many neurodegenerative diseases are associated with the inappropriate activation of neuronal cell death. Excess cell death is limited by a variety of anti-apoptotic proteins, including members of the Bcl-2 family, such as Bcl-2, Bcl-xL, Mcl-1, and A1. When apoptosis is inappropriately suppressed, cells may hyperproliferate. The inappropriate suppression of apoptosis frees neoplastic cells from the regulatory constraints typically imposed on normal proliferating cells. Many chemotherapeutic agents act by inducing apoptosis in proliferating neoplastic cells, but their therapeutic value is limited by the extent to which they are toxic to normal cells. Survival promoting factors and anti-apoptotic agents can modulate the radio- and/or chemosensitivity of human cells.

Many types of chemotherapy suppress hematopoiesis and induce cell death in normal blood cells, which present a dose-limiting side effect of chemotherapy. These adverse side effects can lead to a variety of negative clinical outcomes, including low neutrophil counts that are often associated with fever, a condition known as febrile neutropenia. A patient on chemotherapy who presents with fever and a reduced neutrophil count is typically admitted to the hospital for intravenous antibiotic therapy to limit the risk of infection. Administration of human granulocyte macrophage colony stimulating factor (hGM-CSF) is used to promote the proliferation and maturation of neutrophils, eosinophils, and macrophages from bone marrow progenitors. It also acts as a growth factor for erythroid and megakaryocyte progenitors. The efficacy of hGM-CSF is limited by the blocking effects of many anticancer drugs. These drugs inhibit the cell survival-promoting signals transduced by the GM-CSF receptor. Improved therapeutic methods to offset the toxic effects of chemotherapeutics are required.

### SUMMARY OF THE INVENTION

The present invention provides compositions that enhance the cell survival, inhibit apoptosis in a cell at risk of cell death, or promote cell growth or proliferation, and methods for the therapeutic use of such compositions for the treatment of a subject in need thereof. Such compositions include chimeric polypeptides comprising at least a GM-CSF receptor ligand and an anti-apoptotic moiety (e.g., a Bcl-2 family member). Bcl-2 polypeptides include Bcl-2, Bcl-xL, Mcl-1, and A1. Such compositions are useful for the treatment of human or veterinary subjects. In particular, the compositions and methods described herein are useful for the treatment of virtually any disease or disorder currently treated by administering GM-CSF.

In one aspect, the invention features an isolated chimeric polypeptide containing a GM-CSF receptor ligand and a Bcl-xL polypeptide, where the chimeric polypeptide specifically binds a GM-CSF receptor and enhances cell survival. In one embodiment, the GM-CSF receptor ligand is at least a fragment of GM-CSF or of a GM-CSF receptor antibody. In another embodiment, the chimeric polypeptide contains a ratio of Bcl-XL to GM-CSF that is at least 1:1, 1:2, or 1:3.

In another aspect, the invention features an isolated chimeric polypeptide containing a GM-CSF polypeptide and a Bcl-xL polypeptide, where the chimeric polypeptide specifically binds a GM-CSF receptor and enhances cell survival or promotes cell proliferation.

In yet another aspect, the invention features an isolated nucleic acid molecule that encodes a chimeric polypeptide of any previous aspect. In one embodiment, the chimeric polypeptide contains a full length Bcl-xL or a fragment thereof that enhances cell survival or promotes cell proliferation. In one embodiment, the nucleic acid molecule has substantial nucleic acid sequence identity (e.g., 80%, 85%, 90%, 95%) to SEQ ID NO: 10.

In a related aspect, the invention features an isolated polynucleotide capable of encoding a polypeptide having substantial sequence identity to SEQ ID NO: 1, where the polypeptide enhances cell survival, promotes cell proliferation, or inhibits apoptosis.

In yet another related aspect, the invention features a vector containing a nucleic acid molecule that encodes a polypeptide of any previous aspect. In one embodiment, the vector is an expression vector (e.g., a viral or non-viral expression vector). In another embodiment, the viral expression vector is derived from an adenovirus, retrovirus, adeno-associated virus, herpesvirus, vaccinia virus or polyoma virus. In yet another embodiment, the encoded polypeptide is a fusion polypeptide containing SEQ ID NO:1. In yet another embodiment, the fusion polypeptide contains an affinity tag or a detectable amino acid sequence.

In another aspect, the invention features a host cell containing the vector of any previous aspect, wherein the cell is a mammalian (e.g., human or animal) cell that is in vitro, in vivo, or ex vivo. In one embodiment, the cell is selected from the group consisting of a hematopoietic cell, a dendritic cell, a neuronal cell, and a stem cell. In another embodiment, the cell is at risk of undergoing apoptosis. In still other embodiments, the apoptosis is related to hypoxia, ischemia, reperfusion, stroke, Parkinson's disease, Lou Gehrig's disease, Huntington's chorea, spinal muscular atrophy, spinal chord injury, receipt of a stem cell transplantation, receipt of chemotherapy, or receipt of radiation therapy.

In another aspect, the invention features a pharmaceutical composition containing an effective amount of a chimeric polypeptide of a previous aspect, or fragments thereof, in a pharmaceutically acceptable excipient.

In yet another aspect, the invention features a pharmaceutical composition containing an effective amount of a nucleic acid molecule encoding a chimeric polypeptide of any previous aspect in a pharmaceutically acceptable excipient. In one embodiment, the pharmaceutical composition of a previous aspect further contains an agent selected from the group consisting of a chemotherapeutic agent, radiation agent, hormonal agent, biological agent, an anti-inflammatory agent, an agent that enhances dopamine production, an anticholinergic, a dopamine mimetic, amantadine, an antithrombotic, and a thrombolytic.

In another aspect, the invention features a method of enhancing cell survival, the method involves contacting a cell at risk of cell death with a chimeric polypeptide of a previous aspect, where the contacting enhances cell survival or promotes cell growth.

In another aspect, the method of inhibiting apoptosis in a cell at risk thereof, the method involves contacting the cell at risk of cell death with a chimeric polypeptide of any previous aspect, where the contacting inhibits apoptosis or enhances cell proliferation.

In another aspect, the method involves contacting a cell at risk of cell death with a nucleic acid molecule of a previous aspect, where the contacting enhances cell survival, promotes cell proliferation, or inhibits apoptosis. In one embodiment, the contacting reduces the risk of cell death or enhances cell proliferation by at least 15%. In another embodiment, the GM-CSF receptor ligand is at least a fragment of a GM-CSF polypeptide that binds a GM-CSF receptor or is a fragment of a GM-CSF receptor antibody that enhances cell growth or survival by binding to a GM-CSF receptor. In one embodiment, the cell (e.g., a cell in vitro, in vivo, or ex vivo) is selected from the group consisting of a hematopoietic cell, a dendritic cell, a neuronal cell, and a stem cell. In another embodiment, the cell is at risk of cell death or apoptosis, such as apoptosis associated with hypoxia, ischemia, reperfusion, stroke, Parkinson's disease, Lou Gehrig's disease, Huntington's chorea, spinal muscular atrophy, spinal chord injury, receipt of a stem cell transplantation, receipt of chemotherapy, or receipt of radiation therapy.

In another aspect, the invention features a method of enhancing cell survival in a subject (e.g., a human or veterinary subject) diagnosed as having a disease or disorder characterized by cell death, the method involves administering to the subject a chimeric polypeptide of a previous aspect in an amount effective to enhance cell survival or proliferation.

In another aspect, the method involves enhancing cell survival in a subject (e.g., a human or veterinary subject) diagnosed as having a disease or disorder characterized by cell death, the method involves administering to the subject a nucleic acid molecule encoding the chimeric polypeptide of any previous aspect in an amount effective to enhance cell survival or proliferation. In one embodiment, the nucleic acid encoding the chimeric polypeptide is under the control of a heterologous promoter. In another embodiment, the chimeric polypeptide is produced from an expression construct (e.g., a viral or non-viral expression construct, such as an adenovirus, retrovirus, adeno-associated virus, herpesvirus, vaccinia virus or polyoma virus).

In another aspect, the invention features a method of assessing the efficacy of a cell survival enhancing treatment in a subject. The method involves determining one or more pre-treatment phenotypes; administering a therapeutically effective amount of a chimeric polypeptide of any previous aspect, or a nucleic acid molecule encoding the polypeptide to the subject; and determining the one or more phenotypes after an initial period of treatment with the an apoptosis inhibitor; where the modulation of the one or more phenotypes indicates efficacy of a an apoptosis inhibitor treatment.

In another aspect, the invention features a method of selecting a subject having a disease or disorder characterized by cell death for treatment with an apoptosis inhibitor. The method involves determining one or more pre-treatment phenotypes; administering a therapeutically effective amount of a chimeric polypeptide of a previous aspect, or a nucleic acid molecule encoding the polypeptide to the subject; and determining the one or more phenotypes after an initial period of treatment with the an apoptosis inhibitor, where the modulation of the one or more phenotype is an indication that the disorder is likely to have a favorable clinical response to treatment with a an apoptosis inhibitor. In one embodiment, the decrease in apoptosis, increase in cell survival, or increase in proliferation indicates that the treatment is efficacious. In another embodiment, the method involves obtaining a biological sample from a subject and determining the subject's phenotype after a second period of treatment with the apoptosis inhibitor. In another embodiment, the method further involves obtaining a second biological sample from the subject.

In another embodiment, the method further involves monitoring the treatment or progress of the cell or subject. In another embodiment, the method further involves co-administering one or more of a chemotherapeutic agent (e.g., tamoxifen, trastuzamab, raloxifene, doxorubicin, fluorouracil/5-fu, pamidronate disodium, anastrozole, exemestane, cyclophos-phamide, epirubicin, letrozole, toremifene, fulvestrant, fluoxymester-one, trastuzumab, methotrexate, megastrol acetate, docetaxel, paclitaxel, testolactone, aziridine, vinblastine, capecitabine, goselerin acetate, zoledronic acid, taxol, vinblastine, and vincristine), radiation agent, hormonal agent, biological agent, an anti-inflammatory agent, an agent that enhances dopamine production, an anticholinergic, a dopamine mimetic, amantadine, an antithrombotic, and a thrombolytic to the subject. In another embodiment, the method further involves comparing one or more of the pre-treatment or post-treatment phenotypes to a standard phenotype, where the standard phenotype is the corresponding phenotype in a reference cell (e.g., a cell from the subject, such as hematopoietic cell, an epithelial cell, a bone marrow cell, a hematopoietic stem cells, a neuron, a neural stem cell, an astrocyte, a fibroblast, an endothelial cell, and an oligodendrocyte; or cultured cells, such as cultured cells from the subject, or cells from the subject pre-treatment) or a population of cells. In one embodiment, the sample is one or more of a tissue sample, blood, sputum, bronchial washings, biopsy aspirate, ductal lavage, or nervous tissue biopsy.

In another aspect, the invention features a method of expanding hematopoietic stem cells or progenitor cells by contacting the cells with an effective amount of a polypeptide or nucleic acid molecule of a previous aspect.

In one embodiment of any previous aspect, the chimeric polypeptide inhibits apoptosis. In yet another embodiment of any previous aspect, the polypeptide enhances survival of a cell selected from the group consisting of a hematopoietic cell, a dendritic cell, a neuronal cell, and a stem cell. In another embodiment, the cell is in vitro or in vivo. In other embodiments of a previous aspect, the polypeptide contains full length Bcl-xL or at least a fragment of Bcl-xL capable of inhibiting apoptosis in a cell, such as a fragment that includes or consists of the amino acid sequence GVVLLGSLFSRK; FELRYRRAFS; or SAINGNPSWHLADSPAVNGATG. In yet another embodiment, the polypeptide contains at least a fragment of a GM-CSF polypeptide that binds a GM-CSF receptor, such as a fragment that includes or consists of the amino acid sequence APARSPSPSTQPWEHVNAIQEARRLLNLSRDTAAEMNETVEVISEMFDLQEPT CLQTRLELYKQGLRGSLTKLKGPLTMMASHYKQHCPPTPETSCATQTITFESF KENLKDFLLVIPFDCWEPVQE; EARRLLNLSRD; and TMMASHYKQHCPPTPET, wherein the fragment binds a GM-CSF receptor or has a GM-CSF biological activity. In still other embodiments of any previous aspect, the polypeptide further contains a domain (e.g., a TAT domain) that enhances transport of the polypeptide across the blood-brain barrier. In yet another embodiment, the polypeptide has at least 80%, 90%, or 95% amino acid sequence identity to a GM-CSF-BCL-XL amino acid sequence (SEQ ID NO:1). In yet other embodiments of a previous aspect, the polypeptide contains an alteration (e.g., an insertion, deletion, missense, or nonsense mutation in the amino acid sequence of a GM-CSF or Bcl-XL polypeptide relative to a reference sequence) that enhances protease resistance or that facilitates dimer formation. In yet another embodiment, the polypeptide contains a GM-CSF polypeptide and a Bcl-xL polypeptide. In yet another embodiment, the polypeptide is a fusion protein. In still other embodiments of a previous aspect, the polypeptide contains an affinity tag or a detectable amino acid sequence.

### BRIEF DESCRIPTION OF THE DRAWINGS

FIGS. 1A-1C illustrate the construction, expression and activity of the GM-CSF-Bcl-XL chimeric protein. FIG. 1A is a schematic diagram illustrating the construction of an expression vector encoding the GM-CSF-Bcl-XL chimeric protein. A cDNA encoding human GM-CSF, which was digested with Nde I and Bam HI, was fused with the cDNA encoding human full length Bcl-XL, which was digested with Bgl II and Eco RI. The ligation of the two cDNAs introduced a glycine, serine and threonine linker between the two proteins. The fusion genes were inserted in the E. coli vector pET28b (+) which includes a sequence that encodes a His tag sequence at the N-terminus of the GM-CSF-Bcl-XL cDNA. FIG. 1B shows protein purified on an SDS-PAGE (4-20%) that was visualized by Coomassie brilliant blue staining. Western blot analysis was conducted using an anti His tag monoclonal antibody.

FIGS. 2A-2C show the effect of GM-CSF-Bcl-XL on human blood mononuclear cells. Macrophage/monocytes purified by adhesion from monocytes aphaeresis were treated with human GM-CSF 5 μg/ml, different concentrations of GM-CSF-Bcl-XL, and Lfn-Bcl-XLΔC (30 μg/ml), a chimeric protein that includes the Protective Antigen binding domain of anthrax lethal factor, human Bcl-XL, and the anthrax protective antigen (28 μg/ml), which was incubated in the presence or absence of staurosporine (0.1 μM) and the Jak2 kinase inhibitor TyrAg-490 (0.5 μM), for 72 hours. Cell viability was determined by quantitating the ATP present in metabolically active cells. The mean values were calculated from triplicate measurements. The values are presented as a percentage relative to control cells treated with PBS. FIG. 2B is a graph showing caspase 3/7 activity that was measured in monocyte/macrophages incubated in the presence of cytarabine, daunorubicin, or staurosporine, which are cytotoxic drugs. The cells were also incubated in the presence of GM-CSF-Bcl-XL at different concentrations for 48 hours. A fluorogenic substrate for caspase 3, 1× rodamine 110, bis-(N-CBZ-L-aspartyl-L-glutamyl-L-valyl-L-aspartic acid amide (Z-DEVD-R110) (Songzhu et al., J Biol Chem 275, 288 (2000). was added to each well, and the plate was incubated for 1 hour at room temperature. The fluorescence of each well was measured at an excitation wavelength of 485 nm and an emission wavelength of 535 nm using a Wallack Victor² 1420 Multilabel Counter.

FIGS. 3A and 3B are graphs showing the effect of different recombinant mutants of GM-CSF-Bcl-XL expressed in E. coli. FIG. 3A shows protein synthesis (calculated as a percent of control) in HL-60 cells incubated with 0.1 μM staurosporine (STS) in the presence of the following reagents: STS+human GM-CSF 5 μg/ml; STS+E. coli GM-CSF-BclXL; STS+E. coli GM-CSF-BclXL with a deletion in the C terminus (AC); STS+GM-CSF-Bcl-XLΔL (100 μg/ml); and STS+Bcl-XLΔL-GM-CSF in. The cells were then pulsed with ¹⁴C-leucine for 1 hour and harvested. The leucine incorporation was measured and presented as a percentage relative to PBS-treated cells. The error bars represent the standard error of the mean. FIG. 3B is a graph where cell proliferation in HL-60 cells treated with 0.1 μM staurosporine is measured as leucine incorporation where the cells are incubated with the following reagents: PBS; 0.1 μM staurosporine; 5 μg/ml hGM-CSF; 100 μg/ml hGM-CSF-Bcl-XL (−His tag); 10 μg/ml hGM-CSF-Bcl-XL (−His tag); hGM-CSF-BCl-XL 100 μg/ml (+His tag); hGM-CSF-Bcl-XL 10 μg/ml (+His tag); Lfn-Bcl-XLΔC. The mean values determined from triplicate measurements are plotted versus the leucine incorporation. The error bars represent the standard error of the mean.

FIGS. 4A and 4B are graphs showing the results of a hemopoietic colony assay carried out in the presence or absence of GM-CSF-Bcl-XL. FIG. 4A shows the results of the hemopoietic colony assay using CD34⁺ cells in supplemented media and FIG. 4B shows the results of the assay on cells plated in essential medium. In each case the cells were incubated with different concentration of GM-CSF-Bcl-XL in the presence of cytarabine (right panels). CFU-GM and BFU-E colonies were counted. These results represent the average of colony number from three different experiments. Cultures with CD34⁺ cells alone or with PBS were used to set the value for control growth.

FIG. 5 shows a hemopoietic colony assay carried out in the in the presence or absence of Lfn-Bcl-XL. CD34⁺ cells were plated in supplemented medium and incubated with different concentration of Lfn-Bcl-XL in the presence of cytarabine (right panel). CFU-GM and BFU-E colonies were found only in supplemented medium and they were counted. Results represent the average of colony number from three different experiments. Control cultures with CD34⁺ cells alone or with PBS were used to set the value for normal growth.

FIGS. 6A and 6B are graphs showing the effect of GM-CSF-Bcl-XL on human blood mononuclear cells. In FIG. 6A macrophage/monocytes purified by adhesion from monocytes aphaeresis were treated with the following: human GM-CSF 5 μg/ml; 0.1 mg/ml GM-CSF-Bcl-XL; 0.01 mg/ml GM-CSF-Bcl-XL; or 0.001 mg/ml GM-CSF-Bcl-XL; and a chimeric protein containing the protective antigen binding domain of the anthrax lethal factor (LF) and human Bcl-XL (30 μg/ml) plus the anthrax protective antigen (28 μg/ml) in the presence (black and gray bars) or the absence of staurosporine (0.1 μM) (white bars). In FIG. 6B purified macrophage/monocytes were treated with the following in the absence (white bars) or the presence (striped bars) of the Jak2 kinase inhibitor TyrArg-490 (0.5 μM), for seventy-two hours. The cells were pulsed with ¹⁴C-leucine for 1 hour and harvested. The leucine incorporation was measured and presented as a percentage of the PBS-treated control cells. The mean values were determined from triplicate measurements and were plotted versus the concentration of fusion proteins.

FIGS. 7A, 7B, and 7C are graphs showing cell proliferation (expressed as a percentage of control) in HL-60 cells treated for twenty-four, forty-eight, or seventy-two hours with 5 ug/ml of human GM-CSF, varying concentrations of GM-CSF-Bcl-XL, in the presence or absence of 0.1 μM staurosporine. MTS were added to each well, and the plates were incubated for 1 hour at 37° C. The absorbance at 490 nm was measured using an EIA Multiwell Reader (Sigma Diagnostics) and presented as a percentage relative to PBS-treated cells. The mean values determined from triplicate measurements are plotted versus concentration of fusion protein. The error bars represent the standard error of the mean.

FIG. 8 shows a schematic diagram of the pPICZ-A vector and depicts the expression of the GM-CSF-BCL-XL fusion protein in Pichia pastoris and photographs of a Western blot (left) and an SDS PAGE gel (right). The level of protein expression at twenty-four, forty-eight, and seventy-two hours after induction was monitored by Western blot analysis using an anti-His-Tag antibody. The SDS PAGE gel on the right shows a GM-CSF-Bcl-XL purified protein of the appropriate size visualized with Coomassie brilliant blue.

FIG. 9 is a graph showing the percent of caspase 3/7 activity HL-60 cells incubated for forty-eight hours with the following reagents: PBS (negative control); staurosporine (STS), a pro-apoptotic agent; human GM-CSF 5 μg/ml, STS and human GM-CSF; GM-CSF-Bcl-XL from E. coli 100 μg/ml; STS and E. coli GM-CSF-Bcl-XL; GM-CSF-Bcl-XL from Pichia 100 μg/ml; Pichia GM-CSF-Bcl-XL and STS. A reagent that provides for the detection of caspase 3/7 activity in apoptotic cells (1× Z-DEVD-R110) was added to each well. The plate was then incubated for 1 hour at room temperature. Caspase activity was detected by measuring the fluorescence of each well at an excitation wavelength of 485 nm and an emission wavelength of 535 nm using a Wallack Victor2 1420 Multilabel Counter.

FIGS. 10A and 10B depict the amino acid sequence of a GM-CSF-Bcl-XL chimeric protein and fragments thereof. FIG. 10A provides the sequence of a GM-CSF-Bcl-XL chimeric protein (SEQ ID NO:1). The full-length Bcl-XL portion of the protein is shown in bold. Active fragments of the protein are indicated with underlining (SEQ ID NOS: 2-8). FIG. 10B provides the sequence of another active fragment of GM-CSF (SEQ ID NO:9).

FIGS. 11A and 11B provide nucleic acid sequences. FIG. 11A is the nucleic acid sequence encoding a GM-CSF-BclXL polypeptide (SEQ ID NO:10). The sequence of BclXL is in bold and sequences encoding active fragments of GM-CSF or BclXL are underlined (SEQ ID NOS:11-15). A nucleic acid sequence encoding an extended active fragment of GM-CSF BclXL is shown with gray shading. FIG. 11B is the full length Bcl-XL nucleic acid sequence (SEQ ID NO:16).

FIGS. 12A and 12B provide the vector sequence of pet28b(+) (SEQ ID NO:17) and the vector sequence of pPICZA (SEQ ID NO:18), respectively.

### DETAILED DESCRIPTION OF THE INVENTION

In general, the present invention provides chimeric polypeptides comprising a GM-CSF receptor ligand fused to an anti-apoptotic polypeptide (e.g., a GM-CSF-Bcl-XL chimeric polypeptide) and methods of using these chimeric polypeptides to enhance cell survival or inhibit apoptosis in a cell at risk of cell death. This invention is based, in part, on the discovery that GM-CSF-Bcl-xL chimeric polypeptides are highly effective in reducing apoptosis in cells at risk of undergoing cell death. Accordingly, the invention provides for chimeric polypeptides that include at least a ligand that binds a GM-CSF receptor and an anti-apoptotic moiety.

### Bcl-2 Proteins

Bcl-XL, a member of the Bcl-2 protein family, is able to suppress cell death induced by diverse stimuli in many cell types, including hematopoietic cells. Proteins of the Bcl-2-family are important regulators of programmed cell death. Their function is to integrate survival and death signals that are generated inside and outside cells and to mediate the cell's commitment to cell death. Once a cell is committed to apoptosis, the execution phase begins with the release of cytocrome c from mitochondria and caspase activation. Downstream caspase activation triggers the morphological and biochemical changes associated with efficient cell catabolism. Members of the Bcl-2 family are generally divided into proteins that either promote or inhibit apoptosis. Bcl-XL is a well characterized member of the Bcl-2 family and is able to suppress cell death induced by diverse stimuli in a variety of cell types. Bcl-XL may be delivered to specific target cells via cell surface receptors to prevent cell death. Chimeric proteins containing Bcl-XL fused to the receptor binding domain of different bacterial toxins or to the transduction domain of the HIV TAT protein, rescued neurons in vivo from axotomy, ischemia, and trauma induced cell death.

### Granulocyte Macrophage-Colony Stimulating Factor

Human granulocyte-macrophage colony-stimulating factor (GM-CSF) is a cytokine that promotes the proliferation and maturation of neutrophils, eosinophilis, and macrophages from bone marrow progenitors. Granulocyte macrophage-colony stimulating factor (GM-CSF) was originally discovered because of its ability to stimulate granulocyte and macrophage colony growth from precursor cells in mouse bone marrow (Burgess, A. W. & Metcalf, D. (1980) Blood 56, 947-58). It has subsequently been shown that GM-CSF has other functions associated with its ability to affect the cell number and the activation state of more mature cells such granulocytes, macrophages and eosinophils particularly during immune and inflammatory reactions (Burgess, A. W. & Metcalf, D. (1980) Blood 56, 947-58, Simon et al., (1997) Eur J Immunol 27, 3536-9). The functions of GM-CSF are mediated by binding to a specific receptor comprised of a GM-CSF specific α chain and, in humans, a signal transducing β subunit, which it is shared with IL-3 and IL-5 receptors (Kitamura et al., (1991) Cell 66, 1165-74; Tavernier et al., (1991) Cell 66, 1175-84; Haman et al., (1999) J Biol Chem 274, 34155-63). GM-CSF receptors are found in tissues derived from hematopoietic cells as well as in other cell types, including cells of the nervous system, such as astrocytes, oligodendrocytes, bone marrow derived microglia, and neurons (Sawada, M., Itoh, Y., Suzumura, A. & Marunouchi, T. (1993) Neurosci Lett 160, 131-4).

Clinically, GM-CSF is used to accelerate bone marrow recovery following cancer chemotherapy (Anaissie et al., (1996) Am J Med 100, 17-23; Antman et al., (1988) N Engl J Med 319, 593-8; Vellenga et al., (1996) J Clin Oncol 14, 619-27). GM-CSF can mobilize and induce the maturation of myeloid cells, including monocytes/macrophage and dendritic cells (DCs) (Bernasconi et al. (1995) Int J Cancer 60, 300-7; Melichar, B. & Freedman, R. S. (2002) Int J Gynecol Cancer 12, 3-17). When administered after chemotherapy, GM-CSF reduces the duration of neutropenia and enhances recovery. Other studies have demonstrated that intravenous “priming” with GM-CSF prior to chemotherapy with anthracycline-based chemotherapeutics expands the pool of myeloid progenitor cells and induces quiescence. These effects may enhance myeloprotection and shorten the duration of severe neutropenia induced by chemotherapy (Vadhan-Raj et al. (1992) J Clin Oncol 10, 1266-77). GM-CSF may also stimulate the immune system by enhancing antitumor effects mediated by the innate or adaptive immune systems (Cortes et al., (1998) Leukemia 12, 860-4; Spitler et al., J. (2000) J Clin Oncol 18, 1614-21; Grabstein et al., (1986) Science 232, 506-8). In sum, GM-CSF induces the destruction of tumor cells in vitro by stimulating peripheral blood monocytes (Basak et al., (2002) Blood 99, 2869-79) and enhancing DC maturation (Eager, R. & Nemunaitis, J. (2005) Mol Ther 12, 18-27). GM-CSF has also become an important component of certain vaccine trials (Eager, R. & Nemunaitis, J. (2005) Mol Ther 12, 18-27).

Considerable interest has focused on the use of GM-CSF in stem cell transplantation, either for peripheral blood mobilization of stem cells to allow peripheral blood stem cell collection, or after autologous stem cell transplantation to decrease the duration of neutropenia (Hubel, K., Dale, D. C. & Liles, W. C. (2002) J Infect Dis 185, 1490-50). GM-CSF plays an essential role in the directed differentiation of human embryonic stem (hES) cells into myeloid dendritic cells (DCs). Using a coculture of human stem cells with OP9 stromal cells and then culturing them in a feeder-free culture system in the presence of GM-CSF, the cytokine facilitated the expansion of myeloid lineage cells at various stages of development, including myeloid progenitor and postprogenitor cells. Further culture of myeloid cells in serum-free medium with GM-CSF and IL-4 generated cells that had typical dendritic morphology; expressed high levels of MHC class I and II molecules, CD1a, CD11c, CD80, CD86, DC-SIGN, and CD40, and were capable of antigen (AG) processing, triggering naive T cells in mixed lymphocyte reaction (MLR), and presenting antigens to specific T cell clones through MHC class I proteins (Slukvin et al., (2006) J Immunol 176, 2924-32).

As reported in more detail below, a chimeric protein comprising GM-CSF fused to Bcl-XL was generated to enhance cell survival by reducing apoptosis in cells expressing GM-CSF receptors. The chimeric protein protected cells from staurosporine-induced apoptosis and increased cell proliferation in monocyte cultures. In the presence of TyrAg490, an inhibitor of the Jak2 kinase, GM-CSF-Bcl-XL also promoted proliferation. In contrast, the GM-CSF cytokine alone was completely inhibited by TyrAg490. The chimeric protein is also effective in promoting cell survival in the presence the chemotherapeutics cytarabine and daunorubicin. GM-CSF-Bcl-XL was also able also to promote the differentiation of the CD34⁺ myeloid precursor in the presence of cytarabine and daunorubicin. A fusion protein containing only the Bcl-XL portion did not induce differentiation of CD34+ cells, but was only capable of stimulating proliferation. In sum, under all conditions tested, the antiapoptotic activity of GM-CSF-Bcl-XL was higher than the activity of GM-CSF alone. This indicates that recombinant GM-CSF-Bcl-XL binds the GM-CSF receptor on human monocyte/macrophage cells and bone marrow progenitors and enters into the cells where Bcl-XL blocks cell death and increases cell proliferation and differentiation.

### GM-CSF Receptor Ligands

The GM-CSF receptor ligand includes any polypeptide capable of selectively binding a GM-CSF receptor. While the GM-CSF receptor ligand maybe an endogenous ligand, or a fragment thereof that binds a GM-CSF receptor, the invention is not so limited. The invention encompasses virtually any polypeptide that selectively binds a GM-CSF receptor. A polypeptide that “selectively binds” a GM-CSF receptor is one that binds a GM-CSF receptor, but that does not substantially bind other molecules in a sample, for example, a biological sample. Preferably, a GM-CSF receptor ligand that selectively binds a GM-CSF receptor binds with an affinity constant less than or equal to 10 mM. In various embodiments, the GM-CSF receptor ligand binds the GM-CSF receptor with an affinity constant that is less than or equal to 1 mM, 100 nM, 10 nM, 1 nM, 0.1 nM, or even less than 0.01 or 0.001 nM. In one embodiment, “a GM-CSF receptor” is a polypeptide having substantial identity to GenBank Accession No. NP_{—}000386. GM-CSF receptor ligands include polypeptides that when endogenously expressed bind a naturally occurring GM-CSF receptor, antibodies that bind a GM-CSF receptor, and fragments thereof. In one embodiment, a polypeptide or fragment thereof that binds a naturally occurring GM-CSF receptor is substantially identical to GenBank Accession No. P04141 and binds a GM-CSF receptor.

Antibodies that selectively bind a GM-CSF receptor are useful in the methods of the invention. Preferably, the antibody is fused with a Bcl-XL polypeptide or fragment thereof to form a chimeric polypeptide. Binding to the GM-CSF receptor by this chimeric polypeptide enhances cell survival. Methods of preparing antibodies are well known to those of ordinary skill in the science of immunology. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062, 1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of ˜60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making and unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In one embodiment, an antibody that binds a GM-CSF receptor is monoclonal. Alternatively, the anti-GM-CSF receptor antibody is a polyclonal antibody. The preparation and use of polyclonal antibodies are also known the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing GM-CSF receptors, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding a GM-CSF receptor or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding a GM-CSF receptor, or immunogenic fragments thereof, can be expressed in cells in vitro, followed by isolation of the receptor and administration of the receptor to a suitable host in which antibodies are raised.

Alternatively, antibodies against a GM-CSF receptor may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

### Anti-Apoptotic Moieties

A GM-CSF receptor ligand is fused with an anti-apoptotic moiety to form a chimeric polypeptide. As described herein, such fusions may be made by creating a transcription fusion that encodes a single chimeric polypeptide that includes an anti-apoptotic moiety and a GM-CSF receptor ligand. Alternatively, the GM-CSF receptor ligand and the anti-apoptotic moiety may be expressed from separate expression cassettes that may be included on the same or different expression vectors. Where the two peptides are separately expressed, it is desirable to include a dimerization domain that provides for their association. In one embodiment, the dimerization domain is an amino acid sequence that is appended at the amino or carboxy terminus of the peptide, such that the sequence facilitates the association of the GM-CSF receptor ligand and the anti-apoptotic moiety. Preferably, each of the GM-CSF receptor ligand and the anti-apoptotic moiety includes is an amino acid sequence (e.g., 5, 10, 20, 30, 40, 50, 75, or 100 amino acids in length) that provides for oligomerization in vitro or in vivo. In one embodiment, the dimerization domain is a coiled coil domain that provides for the association of the GM-CSF receptor ligand and the anti-apoptotic moiety. Such tags are known to one skilled in the art of protein engineering and are described, for example, in U.S. Pat. No. 6,911,205; by Liu et al., PNAS, 101:16156-16161; and by Zhang et al., Curr. Biol. 9:417-420, 1999. Exemplary coiled coil domains include heterodimerizing leucine zipper coiled coil system. Dimerization of leucine zippers occurs via the formation of a short parallel coiled coil, with a pair of .alpha.-helices wrapped around each other in a superhelical twist (Zhu et al. J. Mol. Biol. 300:1377-1387, 2000). These coiled-coil structures, termed “leucine zippers,” because of their preference for leucine in every 7th position, have also been used to mediate dimerization in other proteins including antibodies (Hu et al. Science 250:1400-1403, 1990; Blondel and Bedouelle, Protein Eng. 4:457, 1991). Several species of leucine zippers have been identified as particularly useful for dimeric and tetrameric antibody constructs (Pluckthun and Pack Immunotech. 3:83-105, 1997; Kostelny et al. J. Immunol. 148:1547-1553, 1992). Dimerization domains are known in the art and described, for example, at U.S. Pat. Nos. 6,790,624, 6,495,346, 6,486,303, 5,322,801, and at U.S. Patent Publication Nos. 20050106667, 20050003431, 20030077739, 20030054409, 20020037999 In another embodiment, the dimerization domains are oppositely charged polyionic fusion peptide that also contain a cysteine residue that provides for sulfhydryl bond formation. For example, Kleinschmidt et al. (J. Mol. Biol. 327:445-452, 2003) and Richter et al. (Protein Engineering 14:775-783, 2001) describe polyionic adapter peptides, such as Ala-Cys-Glu₈ and Ala-Cys-Lys₈ that provide for the heterodimerization of peptides to which they are appended. Alternatively, more than one such domain may be included in each peptide, such to allow peptides to form multivalent complexes, as described by Deyev et al. (Nature Biotech 21:1486-1492, 2003). Deyev et al. describe the use of barnase and barstar modules to provide for the purification and assembly of oligomeric proteins. In another approach, the association of an anti-apoptotic moiety and a GM-CSF receptor ligand is facilitated by the avidin/biotin system, as described by Asai et al., (Biomol. Eng. 21:145, 2005), where a biotinylated fusion protein binds an avidin conjugated fusion protein. In yet another approach, Asai et al. (J. of Immunol. Methods 299:63-76, 2005) describe methods for protein dimerization that rely on peptides derived from human ribonuclease 1. In this approach, a fifteen amino acid peptide derived from human ribonuclease 1 (human S tag) is appended to a first protein, and residues 21-124 of human ribonuclease 1 are appended to a second protein, such that the dimerization of the two proteins is facilitated by the human ribonuclease amino acid sequences.

Exemplary anti-apoptotic moieties include Bcl-2 family members or fragments thereof. Proteins of the Bcl-2 family are key regulators of programmed cell death in multicellular organisms. Some members of this family, including Bax, Bak, Bok/Mtd, Bad, Bik/Nbk, Bid, Blk, Bim/Bod, and Hrk promote apoptosis, whereas others, including Bcl-2, Bcl-x_{L}, Bcl-w, Bfl-1/A1, Mcl-1, and Boo/Diva inhibit apoptosis. These proteins share one to four conserved Bcl-2 homology domains (BH) designated BH1, BH2, BH3, and BH4. In addition, Bcl-2 family members may possess a C-terminal hydrophobic amino acid sequence that helps localize them to intracellular membranes, primarily the outer mitochondrial membrane (Gross et al., Genes Dev. 13:1899-1911, 1999; Adams et al., Science 281:1322-1326, 1998). The activity of Bcl-2 family proteins can be modulated not only at the transcriptional level but also by post-translational modifications that cleave Bcl-2, Bcl-x_{L}, Bid, Bax, and Bad producing C-terminal fragments with potent pro-apoptotic activity (Basañez et al., J. Biol. Chem., 276: 31083-31091, 2001). In one embodiment, Bcl-2 protein fragments useful in the methods of the invention lack the pro-apoptotic C-terminal.

Bcl-xL

Bcl-xL functions as a Bcl-2-independent regulator of apoptosis. BCL-xL localizes to the outer mitochondrial membrane and has been suggested to protect cells from death by regulating export of ATP from mitochondria and/or by blocking the activation of proapoptotic Bcl-2-related proteins (Basañez et al., J. Biol. Chem. 277, 49360-49365 (2002); Vander Heiden et al., Proc. Natl. Acad. Sci. USA 97, 4666-4667 (2000); Zong et al., Genes Dev. 15, 1481-1486 (2001)). Alternative splicing of a Bcl-xL encoding gene (e.g., GenBank Accession No. Z23115) resulted in 2 distinct BCLX mRNAs (Boise et al., Cell 74: 597-608, 1993). The protein product of the larger mRNA (Bcl-xL) was similar in size and predicted structure to Bcl-2, and it inhibits cell death upon growth factor withdrawal at least as well as BCL2 (Boise et al., Cell 74: 597-608, 1993). Bcl-xL polypeptides have substantial sequence identity to GenBank Accession No. Q07817 and are capable of modulating apoptosis. Preferably, a Bcl-xL polypeptide of the invention reduces apoptosis.

Mcl-1

Other anti-apoptotic Bcl-2 family members useful in the methods of the invention include Mcl-1 and A1. MCL1 was isolated from the ML-1 human myeloid leukemia cell line (Kozopas, et al., Proc. Nat. Acad. Sci. 90: 3516-3520, 1993). Expression of MCL1 increased early in the induction, or programming, of differentiation in ML-1 before the appearance of differentiation markers and mature morphology. MCL1 shows sequence similarity, particularly in the carboxyl portion, to BCL2. Yeast 2-hybrid analysis showed that the full-length 350-amino acid MCL1 protein (MCL1L) interacts with proapoptotic Bcl-2 family proteins and inhibits apoptosis (Bae et al., J. Biol. Chem. 275: 25255-25261, 2000). A 271-amino acid variant that lacks Bcl-2 homology domains 1 and 2 and the transmembrane domain lacks this anti-apoptotic activity (Bae et al., J. Biol. Chem. 275: 25255-25261, 2000). Fragments of an MCL1 protein that are useful in the methods of the invention preferably include at least one Bcl-2 homology domain and are capable of reducing apoptosis.

A1

A1 is another Bcl-2 family member that has anti-apoptotic activity. Lin et al. (J. Immun. 151: 1979-1988, 1993) isolated a novel mouse cDNA sequence, designated BCL2-related protein A1 (Bcl2a1), and identified it as a member of the BCL2 family of apoptosis regulators. The BCL2A1 gene has also been referred to as BCL2L5, BFL1, and GRS. Preferably, A1 is substantially identical to the amino acid sequence of GenBank Accession No. NP_{—}004040. The peptide sequence of A1 contains a region of 80 amino acids that show similarity to Bcl-2 and to the Bcl-2-related gene, MCL1 (Lin et al., J Immunol. 151(4):1979-88, 1993). Preferably, an anti-apoptotic moiety of the invention includes at least a fragment of this region.

In one embodiment, an anti-apoptotic moiety includes at least a fragment of a Bcl-2 family member, wherein the fragment is capable of enhancing cell survival. By “enhances cell survival” is meant increases (e.g., by at least 10%, 20%, 30%, or by as much as 50%, 75%, 85% or 90%) the probability that a cell at risk of cell death will survive. Alternatively, the fragment is capable of inhibiting apoptosis. By “enhances cell proliferation” is meant increases (e.g., by at least 10%, 20%, 30%, or by as much as 50%, 75%, 85% or 90%) the growth or proliferation of a cell. By “inhibits cell death” is meant reduces (e.g., by at least 10%, 20%, 30%, or by as much as 50%, 75%, 85% or 90%) the probability that a cell at risk of cell death will undergo apoptotic, necrotic, or any other form of cell death.

### GM-CSF-Bcl-XL Chimeric Polypeptides and Analogs

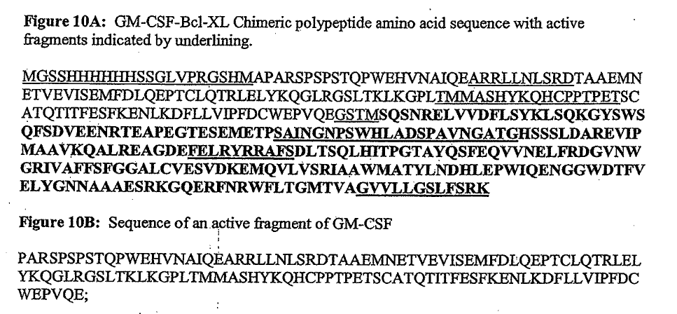

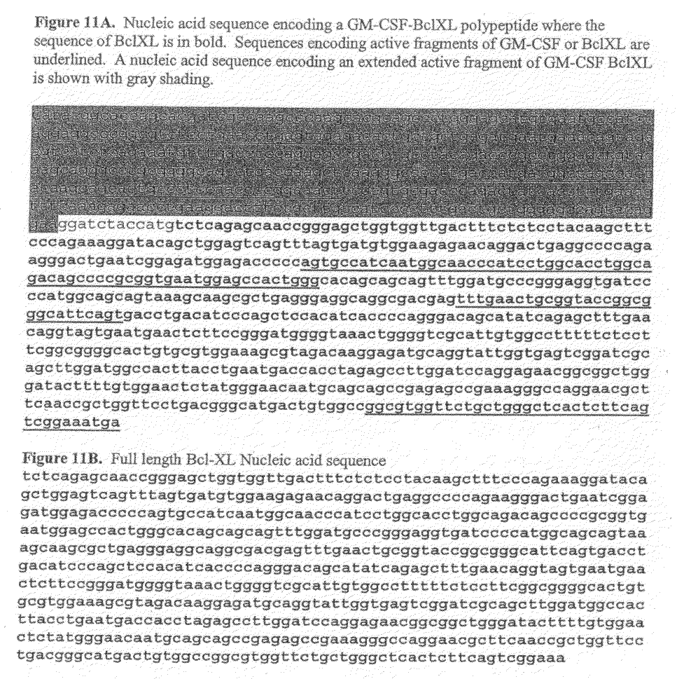

The invention provides for a chimeric polypeptide comprising at least a GM-CSF receptor ligand and an anti-apoptotic moiety. In one embodiment, a chimeric polypeptide comprises a GM-CSF receptor ligand and a Bcl-xL moiety. A “GM-CSF-Bcl-XL chimeric polypeptide” is a polypeptide that comprises at least a fragment of a GM-CSF polypeptide and a fragment of a Bcl-xL polypeptide, where the chimeric polypeptide binds a GM-CSF receptor and enhances cell survival, promotes cell proliferation, or reduces apoptosis. The sequence of an exemplary GM-CSF-Bcl-xL chimeric polypeptide is provided at FIG. 10A. The sequence of GM-CSF-Bcl-xL chimeric polypeptide fragments are shown in FIG. 10A (by underlining) and in FIG. 10B. The sequence of exemplary nucleic acid molecules encoding such polypeptides is provided at FIG. 11A.

The invention includes, but is not limited to chimeric polypeptides comprising one GM-CSF receptor ligand and one anti-apoptotic moiety. In one embodiment, the chimeric polypeptides comprises at least two moieties each of which is independently capable of binding a GM-CSF receptor. In other embodiments, the chimeric polypeptide comprises at least two moieties, each of which is independently capable of reducing apoptosis. Accordingly, the invention provides chimeric polypeptides containing one, two, three or more GM-CSF receptor ligands for each anti-apoptotic moiety. In other embodiments, the invention provides chimeric polypeptides containing one, two, three or more anti-apoptotic moieties for each GM-CSF receptor ligand. Chimeric polypeptides of the invention include GM-CSF receptor ligand to anti-apoptotic moiety ratios of 1:1, 1:2, 2:1, 1:3, or 3:1.

The GM-CSF receptor ligand may be directly fused to the Bcl-xL moiety or the fusion may be accomplished via a linker. A “linker” is any amino acid sequence that joins at least two amino acid sequences of interest. Linkers may vary widely in length. Desirably, a linker is of a length sufficient to optimize the independent functions of the amino acid sequences that it joins. For example, the linker enhances the anti-apoptotic activity of a Bcl-xL moiety and/or the GM-CSF receptor binding activity of a GM-CSF receptor ligand. If desired, the linker may include a cleavage site that is susceptible to proteolytic cleavage upon internalization. Such a cleavage site is capable of liberating an anti-apoptotic moiety when the linker joining the GM-CSF receptor ligand to the anti-apoptotic moiety is proteolytically cleaved. Alternatively, the linker may include an amino acid residue capable of dimerizing (e.g., a cysteine) with another amino acid residues (e.g., a cysteine).

The organization of exemplary chimeric polypeptides comprising linkers are shown below.

GM-CSF - - - linker-BclxL - - - linker - - - GM-CSF or

GM-CSF - - - linker-BclxL - - - linker - - - GM-CSF - - - linker - - - BclxL.

Alternatively, dimerization is mediated by an amino acid tail that is present at the C or NH terminal end of the chimeric polypeptide.

Also included in the invention are chimeric polypeptides or fragments thereof that are modified in ways that enhance their ability to reduce apoptosis in a cell at risk of undergoing cell death. In one embodiment, the invention provides methods for optimizing a GM-CSF-Bcl-XL chimeric amino acid sequence or nucleic acid sequence by producing an alteration in the sequence. Such alterations may include certain mutations, deletions, insertions, or post-translational modifications. These modifications may be made in either the GM-CSF receptor ligand or in the anti-apoptotic moiety (e.g., Bcl-xL). In one embodiment, the GM-CSF receptor ligand is a GM-CSF receptor ligand analog. A “GM-CSF receptor ligand mimetic” binds a GM-CSF receptor, but need not have structural similarity to an endogenous GM-CSF receptor ligand (e.g., GM-CSF). A Bcl-xL mimetic has the anti-apoptotic activity of Bcl-xL, but need not have structural similarity to Bcl-xL.

In other embodiments, the invention further includes analogs of any naturally occurring polypeptide of the invention. Analogs can differ from a naturally occurring polypeptide of the invention by amino acid sequence differences, by post-translational modifications, or by both. Analogs of the invention will generally exhibit at least 85%, more preferably 90%, and most preferably 95% or even 99% identity with all or part of a naturally occurring amino, acid sequence of the invention. The length of sequence comparison is at least 5, 10, 15 or 20 amino acid residues, preferably at least 25, 50, or 75 amino acid residues, and more preferably more than 100 amino acid residues.

A protein or nucleic acid molecule exhibiting at least 50% identity to a reference amino acid sequence (for example, any one of the amino acid sequences described herein) or nucleic acid sequence (for example, any one of the nucleic acid sequences described herein) is “substantially identical.” Preferably, such a sequence is at least 60%, more preferably 80% or 85%, and most preferably 90%, 95% or even 99% identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes.

In various embodiments, the chimeric polypeptides of the invention are altered to delete, substitute, or modify amino acid residues that are sensitive to serum proteases or that are subject to glycosylation. Methods for identifying protease resistant recombinant proteins are described, for example, by Dear et al., Biochem Biophys Res Commun. 2001 Mar. 9; 281(4):929-35. The altered chimeric protein would contain a GM-CSF receptor ligand or an anti-apoptotic moiety having enhanced resistance to proteolysis or having reduced glycosylation, relative to a corresponding naturally occurring GM-CSF receptor ligand or anti-apoptotic moiety (e.g., Bcl-xL). In other embodiments, a chimeric polypeptide of the invention is altered to contain an amino acid capable of dimerizing with another amino acid of the chimeric polypeptide. In one embodiment, the chimeric polypeptide is altered to include at least one cysteine residue that is capable of forming an internal sulfhydryl bridge with another cysteine residue within the chimeric polypeptide. Anti-apoptotic and multidomain pro-apoptotic Bcl-2 family members that form dimers are known in the art (Degterev Nat. Cell Biol. 3, 173-182, 2001). Chimeric polypeptides capable of forming dimers would be selected to identify those that also have enhanced anti-apoptotic activity. Screening methods to identify chimeric polypeptides having anti-apoptotic activity are known in the art and are described herein in the Examples. In one embodiment, the dimer-forming chimeric polypeptide is produced by chemical synthesis. In another embodiment, the dimer-forming chimeric polypeptide is a recombinant polypeptide expressed by a cell (e.g., a prokaryotic or eukaryotic cell) that expresses a heterologous nucleic acid sequence encoding the chimeric polypeptide. In yet other embodiments, the dimer forming chimeric polypeptides contain one, two, three or more anti-apoptotic moieties for each GM-CSF receptor ligand, or contain one, two, three or more GM-CSF receptor ligand moieties for each anti-apoptotic moiety. In preferred embodiments, dimerization occurs between an anti-apoptotic moiety and another anti-apoptotic moiety, between a GM-CSF receptor ligand and another GM-CSF receptor ligand, or between a GM-CSF receptor ligand and an anti-apoptotic moiety.

Analogs can differ from the naturally occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

Amino acids include naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine, phosphothreonine. An amino acid analog is a compound that has the same basic chemical structure as a naturally occurring amino acid, i.e., a carbon that is bound to a hydrogen, a carboxyl group, an amino group, and an R group (e.g., homoserine, norleucine, methionine sulfoxide, methionine methyl sulfonium), but that contains some alteration not found in a naturally occurring amino acid (e.g., a modified side chain); the term “amino acid mimetic” refers to chemical compounds that have a structure that is different from the general chemical structure of an amino acid, but that function in a manner similar to a naturally occurring amino acid. Amino acid analogs may have modified R groups (for example, norleucine) or modified peptide backbones, but retain the same basic chemical structure as a naturally occurring amino acid. In one embodiment, an amino acid analog is a D-amino acid, a β-amino acid, or an N-methyl amino acid.

Amino acids and analogs are well known in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. In addition to full-length polypeptides, the invention also includes fragments of any one of the polypeptides of the invention. As used herein, the term “a fragment” means at least 5, 10, 13, or 15 amino acids. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein GM-CSF-Bcl-xL analogs having a chemical structure designed to mimic GM-CSF-Bcl-xL functional activity can be administered according to methods of the invention. GM-CSF-Bcl-xL analogs may exceed the physiological activity of the original chimeric polypeptide. Methods of analog design are well known in the art, and synthesis of analogs can be carried out according to such methods by modifying the chemical structures such that the resultant analogs exhibit the cell death modulating activity of a reference GM-CSF-Bcl-xL chimeric polypeptide. By “reference” is meant a standard or control condition. A “reference sequence” is a wild-type sequence (e.g., the amino acid or nucleic acid sequence of an endogenous GM-CSF or Bcl-XL polypeptide). These chemical modifications include, but are not limited to, substituting alternative R groups and varying the degree of saturation at specific carbon atoms of a reference GM-CSF-Bcl-xL polypeptide. Preferably, the chimeric polypeptide analogs are relatively resistant to in vivo degradation, resulting in a more prolonged therapeutic effect upon administration. Assays for measuring functional activity include, but are not limited to, those described in the Examples below.

Chimeric polypeptides (e.g., GM-CSF-Bcl-xL) of the invention are capable of specifically binding any cell that expresses a GM-CSF receptor. Such cells include hematopoietic cells, epithelial cells, bone marrow cells, hematopoietic stem cells, neurons, neural stem cells, an astrocytes, a fibroblasts, endothelial cells, and oligodendrocytes. “Specifically binding” means that cells that do not express a GM-CSF receptor are either not bound or are only poorly bound by the chimeric polypeptide. Methods for assaying binding are known in the art. See, Peter Schuck, Lisa F. Boyd, and Peter S. Andersen Current Protocols in Cell biology, Supplement 22, 17.6.1-17.6.22.

Also included in the methods of the invention are chimeric polypeptides (e.g., GM-CSF-Bcl-xL) containing an affinity tag. An “affinity tag” is any moiety used for the purification of a protein or nucleic acid molecule to which it is fixed. Virtually any affinity tag known in the art may be used in the methods of the invention, including, but not limited to, calmodulin-binding peptide (CBP), glutathione-S-transferase (GST), 6×His, Maltose Binding Protein (MBP), Green Fluorescent Protein (GFP), biotin, Strep II, and FLAG. Also useful in the methods of the invention are chimeric polypeptides containing a detectable amino acid sequence. A “detectable amino acid sequence” is a composition that when linked with the nucleic acid or protein molecule of interest renders the latter detectable, via any means, including spectroscopic, photochemical (e.g., luciferase, GFP), biochemical, immunochemical, or chemical means. For example, useful labels include radioactive isotopes, magnetic beads, metallic beads, colloidal particles, fluorescent dyes, electron-dense reagents, enzymes (e.g., horseradish peroxidase, alkaline phosphatase), biotin, digoxigenin, or haptens.

### Nucleic Acid Molecules Encoding Chimeric Polypeptides

The invention further includes nucleic acid molecules that encode a chimeric polypeptide comprising at least a GM-CSF receptor ligand and an anti-apoptotic moiety. Particularly useful in the methods of the invention are nucleic acid molecules encoding a GM-CSF receptor ligand (e.g., GM-CSF), or a Bcl-2 family polypeptide (e.g., Bcl-xL), or fragments thereof. The sequence of exemplary nucleic acid molecules are provided at FIGS. 11A and 11B. Other nucleic acid sequences useful in the methods of the invention include, but are not limited to the sequence of BCL2-related protein A1, which is provided at GenBank Accession No. NM_{—}004049.2, Bcl-xL (BCL2-like 1), which is provided at GenBank Accession No. NM_{—}001191, and Mcl-1, which is provided at GenBank Accession No. NM_{—}021960.

### Chimeric Polypeptide Expression

In general, chimeric polypeptides of the invention may be produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A host cell is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

A polypeptide of the invention may be produced in a prokaryotic host (e.g., a bacteria, such as E. coli) or in a eukaryotic host (e.g., a yeast, such as Pichia pastoris or Saccharomyces cerevisiae, insect cells, e.g., Sf21 cells, or mammalian cells, e.g., NIH 3T3, HeLa, or preferably COS cells). Expression of proteins in bacterial cells can produce larger quantities for further analysis or antibody production. To express a eukaryotic gene in E. coli, the cDNA of interest is cloned into a plasmid or phage vector (called an expression vector) that contains sequences that drive transcription and translation of the inserted gene in bacterial cells. Inserted genes often can be expressed at levels high enough that the protein encoded by the cloned gene corresponds to as much as 10% of the total bacterial protein. Such proteins are typically expressed under the control of an inducible promoter. Such promoters, which are known in the art include, but are not limited to, the T7 promoter, T7/lacO promoter, PLtetO-1 promoter, and the Plac/ara-1 promoter. The T7 and T7/lacO promoters are subject to induction by IPTG. The PLtetO-1 promoter is a tetracycline-regulated promoter that produces protein when it is “turned on” by tetracycline or anhydrotetracycline. The Plac/ara-1 promoter is based on the lac promoter and is activated by the proteins arabinose and IPTG.

Alternatively, high levels of protein expression can be achieved using appropriate vectors expressed in yeast cells (e.g., S. cerevisiae and P. pastoris). Inducible promoters useful in yeast are known in the art. Such promoters include, but are not limited to, GAL1, which is inducible by galactose, CUP1, which is activated by copper or silver ions added to the medium, MET3, which is inactive in the presence of methionine, the PHO5 promoter, which is induced by low or no phosphate in the medium, and AOX1, which is induced by methanol. If desired, such yeast cells can be genetically engineered to express humanized glycosylated proteins that include glycosylations typically observed in human cells. Such yeast cells are known in the art, and are described, for example by Hamilton et al. (Science. 301:1244-6, 2003) and in U.S. Patent Publication Nos. 20040018590 and 20020137134.

In general, GM-CSF-Bcl-xL chimeric peptides are expressed in any prokaryotic or eukaryotic cells known in the art. Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

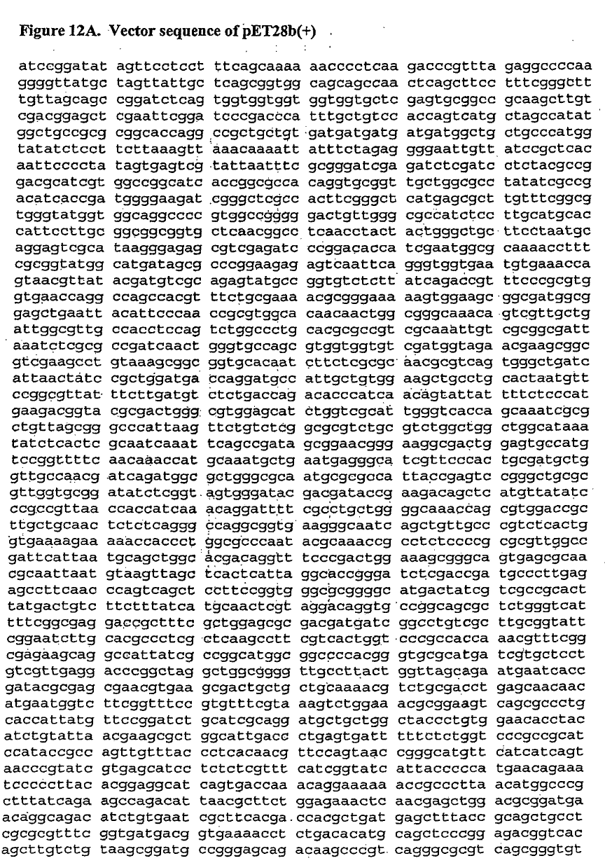

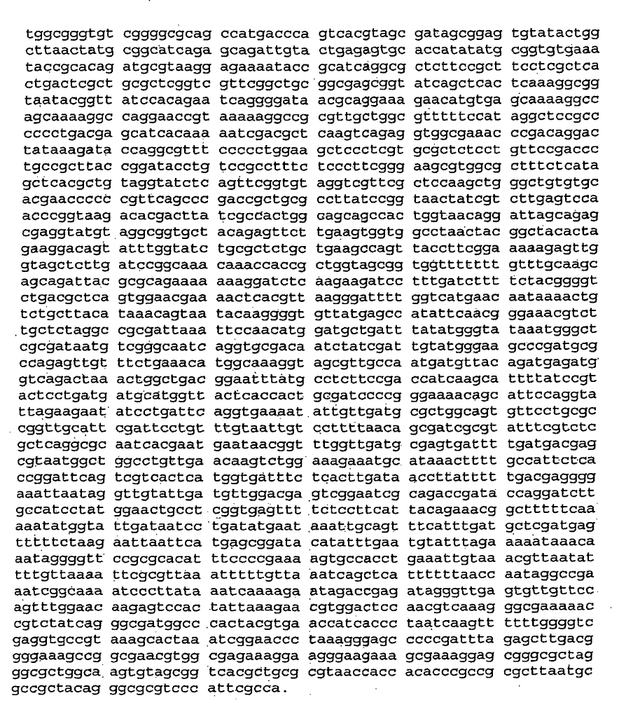

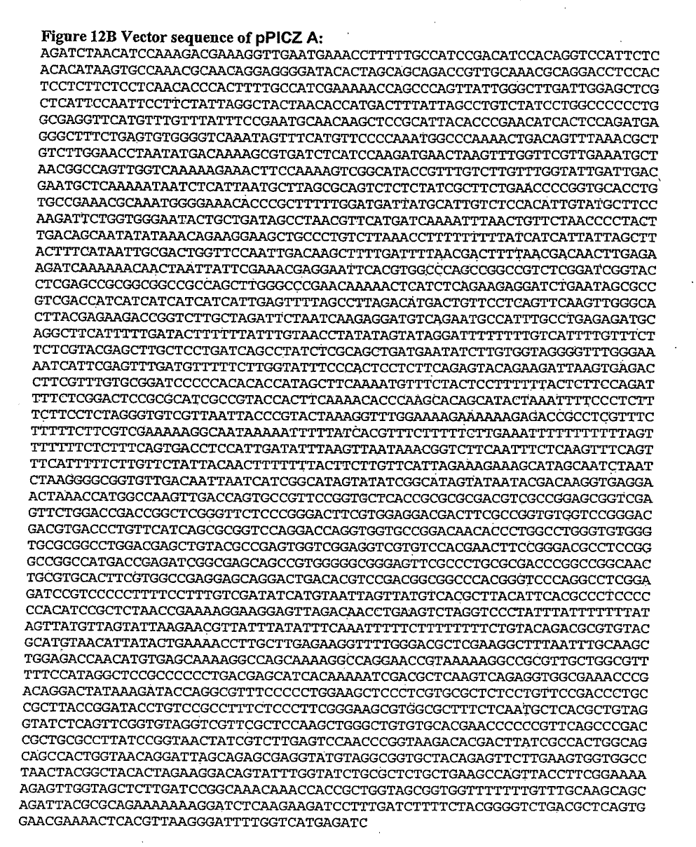

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof. An expression vector is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers. The invention provides for the expression of any of the chimeric polypeptides described herein via an expression vector. The sequence of exemplary expression vectors pET28b(+) and pPICZA is provided in FIGS. 12A and 12B, respectively. In addition, the invention features host cells (e.g., eukaryotic or prokaryotic) comprising a nucleic acid sequence that encodes any chimeric polypeptide described herein.

One particular bacterial expression system for polypeptide production is the E. coli pET expression system (e.g., pET-28) (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Alternatively, recombinant polypeptides of the invention are expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

In another approach, a chimeric polypeptide is produced in a transgenic organism, such as a transgenic plant or animal. By “transgenic” is meant any cell which includes a DNA sequence which is inserted by artifice into a cell and becomes part of the genome of the organism which develops from that cell, or part of a heritable extra chromosomal array. As used herein, transgenic organisms may be either transgenic vertebrates, such as domestic mammals (e.g., sheep, cow, goat, or horse), mice, or rats, transgenic invertebrates, such as insects or nematodes, or transgenic plants.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

In one embodiment, the chimeric polypeptides of the invention are expressed in a transgenic animal, such as a rodent (e.g., a rat or mouse). In addition, cell lines from these mice may be established by methods standard in the art. Construction of transgenes can be accomplished using any suitable genetic engineering technique, such as those described in Ausubel et al. (Current Protocols in Molecular Biology, John Wiley & Sons, New York, 2000). Many techniques of transgene construction and of expression constructs for transfection or transformation in general are known and may be used for the disclosed constructs.

One skilled in the art will appreciate that a promoter is chosen that directs expression of the chosen gene in all tissues or in a preferred tissue. One skilled in the art would be aware that the modular nature of transcriptional regulatory elements and the absence of position-dependence of the function of some regulatory elements, such as enhancers, make modifications such as, for example, rearrangements, deletions of some elements or extraneous sequences, and insertion of heterologous elements possible. Numerous techniques are available for dissecting the regulatory elements of genes to determine their location and function. Such information can be used to direct modification of the elements, if desired. It is desirable that an intact region of the transcriptional regulatory elements of a gene is used. Once a suitable transgene construct has been made, any suitable technique for introducing this construct into embryonic cells can be used.

Animals suitable for transgenic experiments can be obtained from standard commercial sources such as Taconic (Germantown, N.Y.). Many strains are suitable, but Swiss Webster (Taconic) female mice are desirable for embryo retrieval and transfer. B6D2F (Taconic) males can be used for mating and vasectomized Swiss Webster studs can be used to stimulate pseudopregnancy. Vasectomized mice and rats are publicly available from the above-mentioned suppliers. However, one skilled in the art would also know how to make a transgenic mouse or rat. An example of a protocol that can be used to produce a transgenic animal is provided below.

### Production of Transgenic Mice and Rats

The following is but one desirable means of producing transgenic mice. This general protocol may be modified by those skilled in the art.

Female mice six weeks of age are induced to superovulate with a 5 IU injection (0.1 cc, IP) of pregnant mare serum gonadotropin (PMSG; Sigma) followed 48 hours later by a 5 IU injection (0.1 cc, IP) of human chorionic gonadotropin (hCG, Sigma). Females are placed together with males immediately after hCG injection. Twenty-one hours after hCG injection, the mated females are sacrificed by CO.sub.2 asphyxiation or cervical dislocation and embryos are recovered from excised oviducts and placed in Dulbecco's phosphate buffered saline with 0.5% bovine serum albumin (BSA, Sigma). Surrounding cumulus cells are removed with hyaluronidase (1 mg/ml). Pronuclear embryos are then washed and placed in Earle's balanced salt solution containing 0.5% BSA (EBSS) in a 37.5.degree. C. incubator with humidified atmosphere at 5% CO.sub.2, 95% air until the time of injection. Embryos can be implanted at the two-cell stage.

Randomly cycling adult female mice are paired with vasectomized males. Swiss Webster or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos are transferred. After the transferring the embryos, the incision is closed by two sutures.

A desirable procedure for generating transgenic rats is similar to that described above for mice (Hammer et al., Cell 63:1099-112, 1990). For example, thirty-day old female rats are given a subcutaneous injection of 20 IU of PMSG (0.1 cc) and 48 hours later each female placed with a proven, fertile male. At the same time, 40-80 day old females are placed in cages with vasectomized males. These will provide the foster mothers for embryo transfer. The next morning females are checked for vaginal plugs. Females who have mated with vasectomized males are held aside until the time of transfer. Donor females that have mated are sacrificed (CO.sub.2 asphyxiation) and their oviducts removed, placed in DPBA (Dulbecco's phosphate buffered saline) with 0.5% BSA and the embryos collected. Cumulus cells surrounding the embryos are removed with hyaluronidase (1 mg/ml). The embryos are then washed and placed in EBSs (Earle's balanced salt solution) containing 0.5% BSA in a 37.5.degree. C. incubator until the time of microinjection.

Once the embryos are injected, the live embryos are moved to DPBS for transfer into foster mothers. The foster mothers are anesthetized with ketamine (40 mg/kg, IP) and xulazine (5 mg/kg, IP). A dorsal midline incision is made through the skin and the ovary and oviduct are exposed by an incision through the muscle layer directly over the ovary. The ovarian bursa is torn, the embryos are picked up into the transfer pipet, and the tip of the transfer pipet is inserted into the infundibulum. Approximately 10 to 12 embryos are transferred into each rat oviduct through the infundibulum. The incision is then closed with sutures, and the foster mothers are housed singly.

### Construction of Plant Transgenes

Transgenic plants containing a transgene encoding a chimeric polypeptide described herein are useful for production of recombinant polypeptides. A transgenic plant, or population of such plants, expressing at least one transgene (e.g., a transgene encoding a GM-CSF-Bcl-xL chimeric polypeptide) is useful for the production of chimeric polypeptides. In one embodiment, a chimeric polypeptide is expressed by a stably-transfected plant cell line, a transiently-transfected plant cell line, or by a transgenic plant. A number of vectors suitable for stable or extrachromosomal transfection of plant cells or for the establishment of transgenic plants are available to the public; such vectors are described in Pouwels et al. (supra), Weissbach and Weissbach (supra), and Gelvin et al. (supra). Methods for constructing such cell lines are described in, e.g., Weissbach and Weissbach (supra), and Gelvin et all. (supra).

Typically, plant expression vectors include (1) a cloned plant gene under the transcriptional control of 5′ and 3′ regulatory sequences and (2) a dominant selectable marker. Such plant expression vectors may also contain, if desired, a promoter regulatory region (for example, one conferring inducible or constitutive, pathogen- or wound-induced, environmentally- or developmentally-regulated, or cell- or tissue-specific expression), a transcription initiation start site, a ribosome binding site, an RNA processing signal, a transcription termination site, and/or a polyadenylation signal.

Once the desired nucleic acid sequence is obtained as described herein, it may be manipulated in a variety of ways known in the art. For example, where the sequence involves non-coding flanking regions, the flanking regions may be subjected to mutagenesis. A GM-CSF receptor ligand or an anti-apoptotoic moiety encoding DNA sequence may, if desired, be combined with other DNA sequences in a variety of ways. In its component parts, a DNA sequence encoding GM-CSF receptor ligand and an anti-apoptotoic moiety is combined in a DNA construct having a transcription initiation control region capable of promoting transcription and translation in a host cell.

In general, the constructs will involve regulatory regions functional in plants which provide for modified production of chimeric proteins as discussed herein. The open reading frame coding for the GM-CSF receptor ligand or an anti-apoptotoic moiety or functional fragment thereof will be joined at its 5′ end to a transcription initiation regulatory region. Numerous transcription initiation regions are available which provide for constitutive or inducible regulation.

Regulatory transcript termination regions may also be provided in DNA constructs of this invention. Transcript termination regions may be provided by the DNA sequence encoding a GM-CSF receptor ligand or an anti-apoptotoic moiety or may be derived from any convenient transcription termination region. Importantly, this invention is applicable to dicotyledons and monocotyledons, and will be readily applicable to any new or improved transformation or regeneration method. The expression constructs include at least one promoter operably linked to at least one GM-CSF receptor ligand, anti-apoptotoic moiety, or chimeric polypeptide. An example of a useful plant promoter according to the invention is a caulimovirus promoter, for example, a cauliflower mosaic virus (CaMV) promoter. These promoters confer high levels of expression in most plant tissues, and the activity of these promoters is not dependent on virally encoded proteins. CaMV is a source for both the 35S and 19S promoters.

Examples of plant expression constructs using these promoters are found in Fraley et al., U.S. Pat. No. 5,352,605. In most tissues of transgenic plants, the CaMV 35S promoter is a strong promoter (see, e.g., Odell et al., Nature 313:810, 1985). The CaMV promoter is also highly active in monocots (see, e.g., Dekeyser et al., Plant Cell 2:591, 1990; Terada and Shimamoto, Mol. Genet. 220: 389, 1990). Moreover, activity of this promoter can be further increased (i.e., between 2-10 fold) by duplication of the CaMV 35S promoter (see e.g., Kay et al., Science 236: 1299, 1987; Ow et al., Proc. Natl. Acad. Sci., U.S.A. 84:4870, 1987; and Fang et al., Plant Cell 1:141, 1989, and McPherson and Kay, U.S. Pat. No. 5,378,142). Other useful plant promoters include, without limitation, the nopaline synthase (NOS) promoter (An et al., Plant Physiol. 88: 547, 1988 and Rodgers and Fraley, U.S. Pat. No. 5,034,322), the octopine synthase promoter (Fromm et al., Plant Cell 1: 977, 1989), figwort mosiac virus (FMV) promoter (Rodgers, U.S. Pat. No. 5,378,619), and the rice actin promoter (Wu and McElroy, WO91/09948). Exemplary monocot promoters include, without limitation, commelina yellow mottle virus promoter, sugar cane badna virus promoter, ricetungrobacilliform virus promoter, maize streak virus element, and wheat dwarf virus promoter.

Plant expression vectors may also optionally include RNA processing signals, e.g., introns, which have been shown to be important for efficient RNA synthesis and accumulation (Callis et al., Genes and Dev. 1: 1183, 1987). The location of the RNA splice sequences can dramatically influence the level of transgene expression in plants. In view of this fact, an intron may be positioned upstream or downstream of an MLT polypeptide-encoding sequence in the transgene to modulate levels of gene expression. In addition to the aforementioned 5′ regulatory control sequences, the expression vectors may also include regulatory control regions which are generally present in the 3′ regions of plant genes (Thornburg et al., Proc. Natl. Acad. Sci. U.S.A. 84:744, 1987; An et al., Plant Cell 1:115, 1989). For example, the 3′ terminator region may be included in the expression vector to increase stability of the mRNA. One such terminator region may be derived from the PI-11 terminator region of potato. In addition, other commonly used terminators are derived from the octopine or nopaline synthase signals. The plant expression vector also typically contains a dominant selectable marker gene used to identify those cells that have become transformed. Useful selectable genes for plant systems include genes encoding antibiotic resistance genes, for example, those encoding resistance to hygromycin, kanamycin, bleomycin, G418, streptomycin, or spectinomycin. Genes required for photosynthesis may also be used as selectable markers in photosynthetic-deficient strains. Finally, genes encoding herbicide resistance may be used as selectable markers; useful herbicide resistance genes include the bar gene encoding the enzyme phosphinothricin acetyltransferase and conferring resistance to the broad spectrum herbicide Basta (Frankfurt, Germany).

In addition, if desired, the plant expression construct may contain a modified or fully-synthetic structural chimeric polypeptide encoding sequence that has been changed to enhance the performance of the gene in plants. Methods for constructing such a modified or synthetic gene are described in Fischoff and Perlak, U.S. Pat. No. 5,500,365. It should be readily apparent to one skilled in the art of molecular biology, especially in the field of plant molecular biology, that the level of gene expression is dependent, not only on the combination of promoters, RNA processing signals, and terminator elements, but also on how these elements are used to increase the levels of selectable marker gene expression.

### Plant Transformation

Upon construction of the plant expression vector, several standard methods are available for introduction of the vector into a plant host, thereby generating a transgenic plant. These methods include (1) Agrobacterium-mediated transformation (A. tumefaciens or A. rlzizogenes) (see, e.g., Lichtenstein and Fuller In: Genetic Engineering, vol 6, PWJ Rigby, ed, London, Academic Press, 1987; and Lichtenstein, C. P., and Draper, J. In: DNA Cloning, Vol II, D. M. Glover, ed, Oxford, IRI Press, 1985)), (2) the particle delivery system (see, e.g., Gordon-Kamm et al., Plant Cell 2:603 (1990); or BioRad Technical Bulletin 1687, supra), (3) microinjection protocols (see, e.g., Green et al., supra), (4) polyethylene glycol (PEG) procedures (see, e.g., Draper et al., Plant Cell Physiol. 23: 451, 1982; or e.g., Zhang and Wu, Theor. Appl. Genet. 76: 835, 1988), (5) liposome-mediated DNA uptake (see, e.g., Freeman al., Plant Cell Physiol. 25: 1353, 1984), (6) electroporation protocols (see, e.g., Gelvin et al., supra; Dekeyser et al., supra; Fromm et al., Nature 319: 791, 1986; Sheen Plant Cell 2:1027, 1990; or Jang and Sheen Plant Cell 6:1665, 1994), and (7) the vortexing method (see, e.g., Kindle supra). The method of transformation is not critical to the invention. Any method which provides for efficient transformation may be employed. As newer methods are available to transform crops or other host cells, they may be directly applied. Suitable plants for use in the practice of the invention include, but are not limited to, sugar cane, wheat, rice, maize, sugar beet, potato, barley, manioc, sweet potato, soybean, sorghum, cassava, banana, grape, oats, tomato, millet, coconut, orange, rye, cabbage, apple, watermelon, canola, cotton, carrot, garlic, onion, pepper, strawberry, yam, peanut, onion, bean, pea, mango, citrus plants, walnuts, and sunflower.

The following is an example outlining one particular technique, an Agrobacterium-mediated plant transformation. By this technique, the general process for manipulating genes to be transferred into the genome of plant cells is carried out in two phases. First, cloning and DNA modification steps are carried out in E. coli, and the plasmid containing the gene construct of interest is transferred by conjugation or electroporation into Agrobacterium. Second, the resulting Agrobacterium strain is used to transform plant cells. Thus, for the generalized plant expression vector, the plasmid contains an origin of replication that allows it to replicate in Agrobacterium and a high copy number origin of replication functional in E. coli. This permits facile production and testing of transgenes in E. coli prior to transfer to Agrobacterium for subsequent introduction into plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

In another example, plant cells may be transformed by shooting into the cell tungsten microprojectiles on which cloned DNA is precipitated. In the Biolistic Apparatus (Bio-Rad) used for the shooting, a gunpowder charge (22 caliber Power Piston Tool Charge) or an air-driven blast drives a plastic macroprojectile through a gun barrel. An aliquot of a suspension of tungsten particles on which DNA has been precipitated is placed on the front of the plastic macroprojectile. The latter is fired at an acrylic stopping plate that has a hole through it that is too small for the macroprojectile to pass through. As a result, the plastic macroprojectile smashes against the stopping plate, and the tungsten microprojectiles continue toward their target through the hole in the plate. For the instant invention the target can be any plant cell, tissue, seed, or embryo. The DNA introduced into the cell on the microprojectiles becomes integrated into either the nucleus or the chloroplast. In general, transfer and expression of transgenes in plant cells are now routine for one skilled in the art, and have become major tools to carry out gene expression studies in plants and to produce improved plant varieties of agricultural or commercial interest.

### Transgenic Plant Regeneration

Plant cells transformed with a plant expression vector can be regenerated, for example, from single cells, callus tissue, or leaf discs according to standard plant tissue culture techniques. It is well known in the art that various cells, tissues, and organs from almost any plant can be successfully cultured to regenerate an entire plant; such techniques are described, e.g., in Vasil supra; Green et al., supra; Weissbach and Weissbach, supra; and Gelvin et al., supra. In one particular example, a cloned chimeric polypeptide expression construct under the control of the 35SCaMV promoter and the nopaline synthase terminator and carrying a selectable marker (for example, kanamycin resistance) is transformed into Agrobacterium. Transformation of leaf discs, with vector-containing Agrobacterium is carried out as described by Horsch et al. (Science 227: 1229, 1985). Putative transformants are selected after a few weeks (for example, 3 to 5 weeks) on plant tissue culture media containing kanamycin (e.g. 100 Lg/nlL). Kanamycin-resistant shoots are then placed on plant tissue culture media without hormones for root initiation. Kanamycin resistant plants are then selected for greenhouse growth. If desired, seeds from self-fertilized transgenic plants can then be sowed in a soil-less medium and grown in a greenhouse. Kanamycin-resistant progeny are selected by sowing surfaced sterilized seeds on hormone-free kanamycin-containing media.

Analysis for the integration of the transgene is accomplished by standard techniques (see, for example, Ausubel et al. supra; Gelvin et al. supra). Transgenic plants expressing the selectable marker are then screened for transmission of the transgene DNA by standard immunoblot and DNA detection techniques. Each positive transgenic plant and its transgenic progeny are unique in comparison to other transgenic plants established with the same transgene. Integration of the transgene DNA into the plant genomic DNA is in most cases random, and the site of integration can profoundly affect the levels and the tissue and developmental patterns of transgene expression. Consequently, a number of transgenic lines are usually screened for each transgene to identify and select plants with the most appropriate expression profiles.

### Transgenic Lines are Evaluated for Levels of Transgene Expression.

Expression at the nucleic acid level is determined initially to identify and quantitate plants expressing a chimeric polypeptide of the invention. Standard techniques for expression analysis are employed. Such techniques include PCR amplification assays using oligonucleotide primers designed to amplify only transgene nucleic acid templates and solution hybridization assays using transgene-specific probes (see, e.g., Ausubel et al., supra). Those plants that encode a chimeric polypeptide of the invention are then analyzed for protein expression by Western immunoblot analysis using GM-CSF receptor ligand or anti-apoptotic moiety specific antibodies (see, e.g., Ausubel et al., supra). In addition, in situ hybridization and immunocytochemistry according to standard protocols can be done using transgene-specific nucleotide probes and antibodies, respectively, to localize sites of expression within transgenic tissue.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

### Screening Assays

Binding of a GM-CSF-Bcl-xL chimeric polypeptide to a GM-CSF receptor enhances cell survival in cells at risk of undergoing apoptosis. Based in part on this discovery, compositions of the invention are useful for the high-throughput low-cost screening of candidate compounds and chimeric polypeptide analogs that have increased activity, stability, or the ability to cross the blood brain barrier. In one embodiment, novel GM-CSF receptor ligands are isolated that bind to a GM-CSF receptor. Preferably, these ligands activate the receptor. Such ligands are then fused to a Bcl-xL polypeptide or fragment thereof and assayed for their effect on cell survival or apoptosis. Alternatively, the methods and compositions of the invention are useful for the isolation of candidate compounds that increase the biological activity of a GM-CSF-Bcl-xL chimeric polypeptide described herein. In one embodiment, such a candidate compound promotes cell survival or reduces apoptosis when administered in combination with a chimeric polypeptide described herein.

The effect of chimeric polypeptides or candidate compounds on cell survival is assessed in tissues or cells treated with a pro-apoptotic agent. In one working example, candidate compounds or chimeric polypeptides are added at varying concentrations to the culture medium of cultured cells prior to, concurrent with, or following the addition of a proapoptotic agent. Cell survival is then measured using standard methods. In one example, the level of apoptosis in the presence of the candidate compound is compared to the level measured in a control culture medium lacking the candidate molecule. A compound that promotes an increase in cell survival, a reduction in apoptosis, or an increase in cell proliferation is considered useful in the invention; such a candidate compound may be used, for example, as a therapeutic to prevent, delay, ameliorate, stabilize, or treat the toxic effects of a pro-apoptotic agent, such as a chemotherapeutic. In other embodiments, the candidate compound or chimeric polypeptide prevents, delays, ameliorates, stabilizes, or treats a disease or disorder characterized by excess cell death (e.g., a neurodegenerative disorder) or promotes the survival or proliferation of a cell, tissue, or organ at risk of cell death, such as a bone marrow progenitor cell. Such therapeutic compounds are useful in vivo as well as ex vivo.

In some embodiments, a compound that promotes an increase in the biological activity of a chimeric polypeptide of the invention is considered useful. Such compounds are added in combination with a chimeric polypeptide of the invention and their effect on cell survival or proliferation is measured and compared to the effect of the chimeric polypeptide in the absence of the candidate compound. Again, such a candidate compound may be used, for example, as a therapeutic to promote the survival or proliferation of a cell, tissue, or organ at risk of cell death.

In yet another working example, candidate compounds and chimeric polypeptides are screened for those that specifically bind to a GM-CSF receptor expressed by a cell at risk of apoptosis. The efficacy of such a candidate compound is dependent upon its ability to interact with the GM-CSF receptor, or with functional equivalents thereof. Such an interaction can be readily assayed using any number of standard binding techniques and functional assays (e.g., those described in Ausubel et al., supra). In one embodiment, the compound or chimeric polypeptide is assayed in a cell in vitro for receptor binding and for the promotion of cell survival or proliferation. In another embodiment, the promotion of cell survival depends on the ability of the GM-CSF receptor to activate a GM-CSF receptor signal transduction pathway. Such activation is assayed by identifying an increase in levels of phosphorylated Jak2 and Stat5. In other embodiments, the promotion of cell survival or proliferation depends on the intracellular translocation of the GM-CSF receptor ligand.

In one particular working example, a chimeric polypeptide or candidate compound that binds to a GM-CSF receptor is identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for GM-CSF receptor is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds and chimeric polypeptides identified using such methods are then assayed for their effect on cell survival or proliferation as described herein.

In another example, the compound, e.g., the substrate, is coupled to a radioisotope or enzymatic label such that binding of the compound, e.g., the substrate, to the GM-CSF receptor can be determined by detecting the labeled compound, e.g., ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

In yet another embodiment, a cell-free assay is provided in which a GM-CSF receptor polypeptide or a biologically active portion thereof is contacted with a test compound and the ability of the test compound to bind to the polypeptide thereof is evaluated.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of a test compound to bind to a GM-CSF receptor can be accomplished using real-time Biomolecular Interaction Analysis (BIA) (see, e.g., Sjolander, S. and Urbaniczky, C., Anal. Chem. 63:2338-2345, 1991; and Szabo et al., Curr. Opin. Struct. Biol. 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

It may be desirable to immobilize either the chimeric polypeptide or the candidate compound or its GM-CSF receptor target to facilitate separation of complexed from uncomplexed forms of one or both of the proteins, as well as to accommodate automation of the assay. Binding of a candidate compound or chimeric polypeptide to a GM-CSF receptor, or interaction of a test compound or chimeric polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/GM-CSF-Bcl-XL chimeric polypeptide fusion proteins can be adsorbed onto glutathione sepharose beads (Sigma Chemical, St. Louis, Mo.) or glutathione derivatized microtiter plates, which are then combined with the test compound or the test compound and a sample comprising the GST-tagged GM-CSF-Bcl-XL chimeric polypeptide, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Other techniques for immobilizing a complex of a chimeric polypeptide or test compound and a GM-CSF receptor on matrices include using conjugation of biotin and streptavidin. For example, biotinylated proteins can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, an anti-GM-CSF receptor antibody is identified that reacts with an epitope on the GM-CSF receptor. Methods for detecting binding of a GM-CSF receptor antibody to the receptor are known in the art and include immunodetection of complexes, such as enzyme-linked immunoassays (ELISA). If desired, antibodies that bind a GM-CSF receptor are then tested for the ability to activate the receptor. Antibodies that selectively bind a GM-CSF receptor may be fused with a Bcl-XL peptide of the invention and tested for cell survival promoting activity as described herein.

Alternatively, cell free assays for chimeric polypeptides or candidate compounds can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci 18:284-7, 1993); chromatography (gel filtration chromatography, ion-exchange chromatography); electrophoresis and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., J Mol Recognit 11:141-8, 1998; Hage, D. S., and Tweed, S. A., J Chromatogr B Biomed Sci Appl. 699:499-525, 1997). Further, fluorescence energy transfer may also be conveniently utilized, as described herein, to detect binding without further purification of the complex from solution. Preferably, cell free assays preserve the structure of the GM-CSF receptor, e.g., by including a membrane component or synthetic membrane components.

Compounds, chimeric polypeptides, GM-CSF receptor antibodies, and other GM-CSF receptor ligands isolated by this method (or any other appropriate method) may, if desired, be further purified (e.g., by high performance liquid chromatography). In one embodiment, these candidate compounds are fused with a Bcl-XL polypeptide, or fragment thereof, and the fusion may be tested for its ability to promote cell survival or reduce apoptosis in a cell at risk thereof (e.g., as described herein). Compounds isolated by this approach may also be used, for example, as therapeutics to treat any disease or condition characterized by excess cell death in a subject. A “subject” is typically a mammal in need of treatment, such as a human or veterinary patient (e.g., rodent, such as a mouse or rat, a cat, dog, cow, horse, sheep, goat, or other livestock).

Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized.

In another embodiment, a candidate compound is tested for its ability to enhance the cell survival promoting activity of a GM-CSF-Bcl-XL chimeric polypeptide. The cell survival promoting activity of a GM-CSF-Bcl-XL chimeric polypeptide is assayed using any standard method.

Each of the DNA sequences listed herein may also be used in the discovery and development of a therapeutic compound, such as a chimeric polypeptide, that promotes cell survival.

Small molecules of the invention preferably have a molecular weight below 2,000 daltons, more preferably between 300 and 1,000 daltons, and most preferably between 400 and 700 daltons. It is preferred that these small molecules are organic molecules.

### Test Compounds and Extracts

In general, compounds capable of increasing the activity of a chimeric polypeptide of the invention (e.g., GM-CSF-Bcl-xL) are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to increase the activity of a chimeric polypeptide of the invention, or to binding a GM-CSF receptor, further fractionation of the positive lead extract is necessary to isolate chemical constituents responsible for the observed effect. Thus, the goal of the extraction, fractionation, and purification process is the careful characterization and identification of a chemical entity within the crude extract that increases the activity of a chimeric polypeptide of the invention (e.g., GM-CSF-Bcl-xL). Methods of fractionation and purification of such heterogenous extracts are known in the art. If desired, compounds shown to be useful as therapeutics for the treatment of any disease or condition associated with cell death.

### Cell Survival or Proliferation Enhancing Therapy

Chimeric polypeptides of the invention and related compounds are useful for enhancing the survival or proliferation of virtually any cell type that expresses a GM-CSF receptor. Where a cell that expresses a GM-CSF receptor is at risk of cell death, administration of a chimeric polypeptide described herein is useful for preventing or treating a disease or disorder associated with cell death. In one embodiment, cell death is associated with the toxicity of a medication, such as a chemotherapeutic agent. For example, chimeric polypeptides of the invention are useful to prevent or treat (e.g., ameliorate, stabilize, reverse or slow) the cell death (e.g., apoptotic cell death) of a cell type at risk of undergoing apoptosis in response to a pro-apoptotic event (e.g., chemotherapy, radiation, ischemic injury or a neurodegenerative disease). In one embodiment, the cell at risk of undergoing apoptosis is a monocyte or hematopoetic cell type that is at risk of apoptosis in response to chemotherapy. In other embodiments, methods and compositions of the invention are useful for the treatment or prevention of cell death associated with hypoxia, such as a stroke, ischemic injury, or reperfusion. In other embodiments, the methods and compositions not only reduce cell death but promote cell proliferation.

The chimeric polypeptides of the invention and related compositions are also useful for enhancing the survival or proliferation of a cell in vitro or in vivo. For example, chimeric polypeptides may be administered for the treatment of patients receiving stem cell therapies, or in any patient where it is desirable to increase the survival of a transplanted cell, tissue, or organ. In other embodiments, the methods and compositions of the invention are useful for the ex vivo expansion of a cultured cell, tissue or organ, particularly where the cell is a stem cell or the tissue or organ comprises a stem cell. For example, the invention provides for the expansion of cultures that contain hematological or neuronal stem cells or dendritic cells.

### Pharmaceutical Compositions

The compositions of the invention (e.g., chimeric polypeptides and the nucleic acid molecules encoding them) can be administered in a pharmaceutically acceptable excipient, such as water, saline, aqueous dextrose, glycerol, or ethanol. The compositions can also contain other medicinal agents, pharmaceutical agents, adjuvants, carriers, and auxiliary substances such as wetting or emulsifying agents, and pH buffering agents. Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate dosages is within the skill of one in the art given the parameters herein.

A “therapeutically effective amount” is an amount sufficient to effect a beneficial or desired clinical result. A therapeutically effective amount can be administered in one or more doses. In terms of treatment, an effective amount is an amount that is sufficient to palliate, ameliorate, stabilize, reverse or slow the progression of a disease characterized by cell death, or otherwise reduce the pathological consequences of apoptosis. In another embodiment, an effective amount is an amount sufficient to promote the proliferation or growth of a desirable cell type (e.g. a neuronal cell or a cell at risk of cell death). A therapeutically effective amount can be provided in one or a series of administrations. The effective amount is generally determined by the physician on a case-by-case basis and is within the skill of one in the art.

As a rule, the dosage for in vivo therapeutics or diagnostics will vary. Several factors are typically taken into account when determining an appropriate dosage. These factors include age, sex and weight of the patient, the condition being treated, the severity of the condition and the form of the antibody being administered.

The dosage of the chimeric polypeptide compositions can vary from about 0.01 mg/m² to about 500 mg/m², preferably about 0.1 mg/m² to about 200 mg/m², most preferably about 0.1 mg/m² to about 10 mg/m². Alternatively, the dosages of the chimeric polypeptide compositions can vary from about 0.01 mg/kg per day to about 1000 mg/kg per day. It is expected that doses ranging from about 50 to about 2000 mg/kg will be suitable. In various embodiments, a dosage ranging from about 0.5 to about 100 mg/kg of body weight is useful; or any dosage range in which the low end of the range is any amount between 0.1 mg/kg/day and 90 mg/kg/day and the upper end of the range is any amount between 1 mg/kg/day and 100 mg/kg/day (e.g., 0.5 mg/kg/day and 5 mg/kg/day, 25 mg/kg/day and 75 mg/kg/day).

Administrations can be conducted infrequently, or on a regular weekly basis until a desired, measurable parameter is detected, such as diminution of disease symptoms. Administration can then be diminished, such as to a biweekly or monthly basis, as appropriate.

Compositions of the present invention are administered by a mode appropriate for the form of composition. Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally, alone or in combination with tumoricidal antibodies. Therapeutic compositions of chimeric polypeptides are often administered by injection or by gradual perfusion.

Compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, compositions are preferably supplied in unit dosage form suitable for administration of a precise amount. Also contemplated by this invention are slow release or sustained release forms, whereby a relatively consistent level of the active compound are provided over an extended period.

Another method of administration is intralesionally, for instance by direct injection directly into the apoptotic tissue site; into a site that requires cell growth; or into a site where a cell, tissue or organ is at risk of cell death. Alternatively, the chimeric polypeptide or related compound is administered systemically. For methods of combination therapy comprising administration of a chimeric polypeptide in combination with a chemotherapeutic agent, the order in which the compositions are administered is interchangeable. Concomitant administration is also envisioned.

Methods of the invention are particularly suitable for use in enhancing cell survival or proliferation in the central nervous system (CNS). When the site of delivery is the brain, the therapeutic agent must be capable of being delivered to the brain. The blood-brain barrier limits the uptake of many therapeutic agents into the brain and spinal cord from the general circulation. Molecules which cross the blood-brain barrier use two main mechanisms: free diffusion and facilitated transport. Because of the presence of the blood-brain barrier, attaining beneficial concentrations of a given therapeutic agent in the CNS may require the use of specific drug delivery strategies. Delivery of therapeutic agents to the CNS can be achieved by several methods.

One method relies on neurosurgical techniques. For instance, therapeutic agents can be delivered by direct physical introduction into the CNS, such as intraventricular, intralesional, or intrathecal injection. Intraventricular injection can be facilitated by an intraventricular catheter, for example, attached to a reservoir, such as an Ommaya reservoir. Methods of introduction are also provided by rechargeable or biodegradable devices. Another approach is the disruption of the blood-brain barrier by substances which increase the permeability of the blood-brain barrier. Examples include intra-arterial infusion of poorly diffusible agents such as mannitol, pharmaceuticals which increase cerebrovascular permeability such as etoposide, or vasoactive agents, such as leukotrienes or by convention enhanced delivery by catheter (CED). Further, it may be desirable to administer the compositions locally to the area in need of treatment; this can be achieved, for example, by local infusion during surgery, by injection, by means of a catheter, or by means of an implant, the implant being of a porous, non-porous, or gelatinous material, including membranes, such as silastic membranes, or fibers. A suitable such membrane is Gliadel® provided by Guilford Pharmaceuticals Inc.

Other delivery systems can include time-release, delayed release or sustained release delivery systems. Such systems can avoid repeated administrations of compositions of the invention, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(−)-3-hydroxybutyric acid (European Patent No. 133, 988), copolymers of L-glutamic acid and gamma-ethyl-L-glutamate (Sidman, K. R. et al., Biopolymers 22: 547-556), poly(2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono- di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like. Specific examples include, but are not limited to: (a) erosional systems in which the agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein.

Liposomes are commercially available from Gibco BRL, for example, as LIPOFECTIN™ and LIPOFECTACE™, which are formed of cationic lipids such as N-[1-(2, 3 dioleyloxy)-propyl]-N,N,N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88, 046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/0307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (wherein an agent is dispersed throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and composition are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. E., et al., Biotechnol. Bioeng., 52: 96-101; Mathiowitz, E., et al., Nature 386: 410-414.

Both non-biodegradable and biodegradable polymeric matrices can be used to deliver the compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

A chimeric polypeptide (e.g., GM-CSF-BclxL) disclosed herein may be derivatized by the attachment of one or more chemical moieties to the protein moiety. The chemically modified derivatives may be further formulated for intraarterial, intraperitoneal, intramuscular, subcutaneous, intravenous, oral, nasal, rectal, buccal, sublingual, pulmonary, topical, transdermal, or other routes of administration. Chemical modification of biologically active proteins has been found to provide additional advantages under certain circumstances, such as increasing the stability and circulation time of the therapeutic protein and decreasing immunogenicity. The chemical moieties suitable for derivatization may be selected from among water soluble polymers. The polymer selected should be water soluble so that the protein to which it is attached does not precipitate in an aqueous environment, such as a physiological environment. Preferably, for therapeutic use of the end-product preparation, the polymer will be pharmaceutically acceptable. One skilled in the art will be able to select the desired polymer based on such considerations as whether the polymer/polypeptide conjugate will be used therapeutically, and if so, the desired dosage, circulation time, resistance to proteolysis, and other considerations.

The water soluble polymer may be selected from the group consisting of, for example, polyethylene glycol, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols and polyvinyl alcohol. Polyethylene glycol propionaldenhyde may provide advantages in manufacturing due to its stability in water.

The polymer may be of any molecular weight, and may be branched or unbranched. In one embodiment, the polymer is polyethylene glycol having a preferred molecular weight between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the protein with consideration of effects on functional activity of the protein. In one example, polyethylene glycol may be covalently bound through amino acid residues via a reactive group, such as a free amino or carboxyl group. Reactive groups are those to which an activated polyethylene glycol molecule may be bound. The amino acid residues having a free amino group may include lysine residues and the N-terminal amino acid residues, those having a free carboxyl group may include aspartic acid residues glutamic acid residues and the C-terminal amino acid residue. Sulfhydryl groups may also be used as a reactive group for attaching the polyethylene glycol molecule(s). Preferred for therapeutic purposes is attachment at an amino group, such as attachment at the N-terminus or lysine group. Attachment at residues important for GM-CSF receptor binding should be avoided.

In other embodiments, pharmaceutical compositions of the invention further include cytokines that induce GM-CSF. Such cytokines include, but are not limited to, IL-1β and TNF-α. Such compositions are suitable for use in vivo (e.g., for administration to a subject for the modulation of apoptosis) or for use in vitro (e.g., for the modulation of apoptosis in a cell in vitro).

### GM-CSF-Bcl-XL Expression Therapy

The in vivo or in vitro expression of a GM-CSF-Bcl-XL chimeric polypeptide, or fragment thereof is another therapeutic approach for promoting the survival or proliferation of a cell at risk of undergoing cell death. Nucleic acid molecules encoding chimeric polypeptides of the invention can be delivered to cells of a subject that are at risk for apoptosis. The expression of a chimeric polypeptide in a cell promotes proliferation, prevents apoptosis, or reduces the risk of apoptosis in that cell or in a target cell or tissue. The nucleic acid molecules must be delivered to the cells of a subject in a form in which they can be taken up so that therapeutically effective levels of the chimeric protein can be produced. Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a chimeric protein, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer a chimeric polynucleotide to a target cell, tissue, or systemically.

Non-viral approaches can also be employed for the introduction of a therapeutic to a cell requiring modulation of cell death (e.g., a cell of a patient). For example, a nucleic acid molecule can be introduced into a cell by administering the nucleic acid molecule in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of a chimeric polynucleotide into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant chimeric GM-CSF-Bcl-XL protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

### Methods of Assaying Cell Viability

Chimeric polypeptides, polypeptide analogs, and related compounds that enhance the survival of a cell at risk of cell death are useful as therapeutics in the methods of the invention. Assays for measuring cell growth or viability are known in the art, and are described herein. See also, Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Chimeric polypeptides and candidate compounds that decrease cell death (e.g., by reducing apoptosis) are also useful in the methods of the invention. Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

### Dendritic Cell Vaccines

The invention also provides methods for inhibiting the apoptosis or promoting the proliferation of dendritic cells during the production of a therapeutic or prophylactic vaccine. In general, the vaccine includes a cell (e.g., a dendritic cell) derived from a subject that requires vaccination. In general, the cell is obtained from a biological sample of the subject, such as a blood sample or a bone marrow sample. Preferably, a dendritic cell or dendritic stem cell is obtained from the subject, and the cell is cultured in vitro to obtain a population of dendritic cells. The cultured cells are contacted with an antigen (e.g., a cancer antigen) in the presence of a chimeric polypeptide of the invention. Desirably, a dendritic cell contacted with the antigen in the presence of the chimeric polypeptide is at reduced risk of apoptosis relative to a dendritic cell contacted in the absence of the chimeric polypeptide. Optionally, the contacted cells are expanded in number in vitro. The cells are then re-introduced into the subject where they enhance or elicit an immune response against an antigen of interest (e.g., a cancer antigen). Methods for producing such vaccines are known in the art and are described, for example, by Zhu et al., J Neurooncol. 2005 August; 74(1):9-17; Nair et al., Int. J. Cancer. 1997; 70:706-715; and Fong et al., Annu. Rev. Immunol. 2000; 18:245-273.

Typically vaccines are prepared in an injectable form, either as a liquid solution or as a suspension. Solid forms suitable for injection may also be prepared as emulsions, or with the polypeptides encapsulated in liposomes. The cells are injected in any suitable carrier known in the art. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

Adjuvants are immunostimulating agents that enhance vaccine effectiveness. Effective adjuvants include, but are not limited to, aluminum salts such as aluminum hydroxide and aluminum phosphate, muramyl peptides, bacterial cell wall components, saponin adjuvants, and other substances that act as immunostimulating agents to enhance the effectiveness of the composition.

Vaccines are administered in a manner compatible with the dose formulation. By an effective amount is meant a single dose, or a vaccine administered in a multiple dose schedule, that is effective for the treatment or prevention of a disease or disorder. Preferably, the dose is effective to inhibit the growth of a neoplasm. The dose administered will vary, depending on the subject to be treated, the subject's health and physical condition, the capacity of the subject's immune system to produce antibodies, the degree of protection desired, and other relevant factors. Precise amounts of the active ingredient required will depend on the judgement of the practitioner.

### Use

The methods of the invention provide a means for modulating apoptosis or for enhancing cell proliferation. This modulation can be carried out in vivo or in vitro. For therapeutic uses in vivo, the compositions or agents described herein may be administered systemically, for example, formulated in a pharmaceutically-acceptable buffer such as physiological saline. The compositions and methods of the invention can be used for the treatment of virtually any condition in which the administration of GM-CSF is useful. Such conditions include bone marrow recovery after bone marrow transplantation, coronary artery disease, Crohn's disease, cytotoxic drug treatment, hemodynamic stroke, infectious disease (e.g., HIV, lymphocytic leukemia, mucositis, myeloid engraftment, myelodysplastic syndromes, neutropenia, rheumatoid arthritis, stem cell transplantation (e.g., hematopoietic stem cell transplantation), white blood cell shortages, wound healing. Specifically, compositions of the invention may be used to boost immune systems to fight infections (e.g., AIDS or during transplantation); as Vaccine adjuvants for the treatment of cancer and infectious diseases; to stimulate cell based vaccines; for the treatment of nervous system injuries (traumatic injury, spinal cord injury, ischemic injury, stroke), to stimulate stem cell growth and/or differentiation, to stimulate dendritic cells, to alleviate the symptoms of or shorten the duration of diarrhea and/or mucositis. In preferred embodiments, the compositions of the invention are administered in a form that provides for their delivery across the blood-brain barrier. In the context of treating a neurodegenerative disease, or cell death related to hypoxia, ischemia, reperfusion, stroke, or spinal cord injury a chimeric polypeptide is provided in an amount sufficient to reduce cell death, enhance cell growth, or reduce a symptom associated with the death of a neuronal cell. In the context of treating a bone marrow transplant patient or a stem cell transplant patient, a chimeric polypeptide of the invention is administered in an amount sufficient to enhance survival of a transplanted cell. Typically, the compositions are administered to a patient already suffering from a disease or disorder characterized by cell death, in an amount sufficient to cure or at least partially arrest a symptom associated with cell death or enhance cell growth.

For in vitro uses, cells in culture (e.g., stem cells, neural cells, dendritic cells) are contacted with a chimeric polypeptide of the invention in an amount sufficient to enhance the survival of the cell in vitro. A cell in vitro that is contacted with a chimeric polypeptide of the invention is less likely to undergo apoptosis than a cell cultured under similar conditions but not contacted with a chimeric polypeptide. Advantageously, chimeric polypeptides promote the survival or proliferation of cultured cells and provide for the in vitro expansion of the cultured cells. Optionally, the cultured cells in combination with a chimeric polypeptide are administered to a patient in need thereof.

### Combination Therapies

As described herein, chimeric polypeptides of the invention are useful for reducing apoptosis or promoting proliferation. Accordingly, the compositions of the invention may, if desired, be combined with any standard therapy typically used to treat a disease or disorder characterized by excess cell death. In one embodiment, the standard therapy is useful for the treatment of cell death or apoptosis associated with hypoxia, ischemia, reperfusion, stroke, Alzheimer's disease, Parkinson's disease, Lou Gehrig's disease, Huntington's chorea, spinal muscular atrophy, spinal chord injury, receipt of a stem cell transplantation, receipt of chemotherapy, or receipt of radiation therapy. In particular, for diseases characterized by the death of dopaminergic cells, such as Parkinson's disease, the chimeric polypeptides of the invention may be administered in combination with an agent that enhances dopamine production or a dopamine mimetic, with an antidyskinetic agent, such as amantadine or an anti-cholinergic. For ischemic injuries related to the presence of a thrombosis, a chimeric polypeptide of the invention is administered in combination with an antithrombotic or a thrombolytic agent. Such methods are known to the skilled artisan and described in Remington's Pharmaceutical Sciences by E. W. Martin.

For the treatment of diseases or disorders affecting the central nervous system, the chimeric polypeptides are provided in combination with agents that enhance transport across the blood-brain barrier. Such agents are known in the art and are described, for example, by U.S. Patent Publication Nos. 20050027110, 20020068080, and 20030091640. Other compositions and methods that enhance delivery of an active agent across the blood brain barrier are described in the following publications: Batrakova et al., Bioconjug Chem. 2005 July-August; 16(4):793-802; Borlongan et al., Brain Res Bull. 2003 May 15; 60(3):297-306; Kreuter et al., Pharm Res. 2003 March; 20(3):409-16; and Lee et al., J Drug Target. 2002 September; 10(6):463-7. Other methods for enhancing blood-brain barrier transport include the use of agents that permeabilize tight junctions via osmotic disruption or biochemical opening; such agents include RMP-7 (Alkermes), and vasoactive compounds (e.g., histamine). Other agents that enhance transport across the blood-brain barrier enhance transcytosis across the endothelial cells to the underlying brain cells. Enhanced transcytosis can be achieved by increasing endocytosis (i.e. internalisation of small extracellular molecules) using liposomes or nanoparticles loaded with a drug of interest.

Alternatively, a chimeric polypeptide or other composition of the invention is administered in combination with a chemotherapeutic, such that the chimeric polypeptide reduces the toxic effects typically associated with chemotherapy. For example, a patient that receives a chemotherapeutic and a chimeric polypeptide of the invention is less likely to suffer from side-effects associated with the apoptosis of normal cells (e.g., reduced neutrophil count) than a patient that receives only the chemotherapeutic. A composition of the invention is administered prior to, concurrent with, or following the administration of any one or more of the following: a chemotherapeutic agent, radiation agent, hormonal agent, biological agent, an anti-inflammatory agent. Exemplary chemotherapeutic agents include tamoxifen, trastuzamab, raloxifene, doxorubicin, fluorouracil/5-fu, pamidronate disodium, anastrozole, exemestane, cyclophos-phamide, epirubicin, letrozole, toremifene, fulvestrant, fluoxymester-one, trastuzumab, methotrexate, megastrol acetate, docetaxel, paclitaxel, testolactone, aziridine, vinblastine, capecitabine, goselerin acetate, zoledronic acid, taxol, vinblastine, and vincristine.

In other embodiments, a chimeric polypeptide (e.g., GM-CSF-Bcl-xL) of the invention is provided in combination with a cytokine that upregulates GM-CSF expression (e.g., TNFα, IL-1β).

### Patient Monitoring

The treatment or disease state of a patient administered a composition of the invention that includes a chimeric polypeptide can be monitored by assessing the level of cell death or apoptosis present in a cell, tissue, or organ of the patient. For patient's suffering from a disease or disorder characterized by excess cell death (e.g., a neurodegenerative disease), this monitoring typically involves monitoring the neurological symptoms typically associated with the death of neuronal cells. Neurological symptoms associated with a neurodegenerative disease may include any one or more of the following: apoptosis level; tremors; rigidity; substantia nigra impairment; depression; areflexia; hypotonia; fasciculations; muscle atrophy; involuntary movements of the head, trunk and limbs; mutated survival motor neuron 1 (SMN1) gene; sudden numbness or weakness; sudden confusion; sudden trouble speaking; sudden trouble understanding speech; sudden trouble seeing in one or both eyes; sudden trouble with walking; dizziness; loss of balance; loss of coordination; sudden severe headache of unknown etiology; bradykinesia; postural instability; loss of consciousness; confusion; lightheadedness; dizziness; blurred vision; tired eyes; ringing in the ears; bad taste in the mouth; fatigue; lethargy; an alteration in sleep pattern; behavioral alteration; mood alteration; memory deficit; concentration deficits; attentional deficits; cognitive deficits; vomiting; nausea; convulsions; seizures; inability to awaken; pupil dilation; slurred speech; weakness or numbness in the extremities; restlessness; and agitation. Compositions that produce a reduction in the severity of any one or more of the preceding symptoms are considered useful in the methods of the invention.

For patient's suffering from adverse side-effects associated with the toxic effects of chemotherapy, an effective composition is one that reduces the toxic side-effects of chemotherapy. Typically, the efficacy of the composition in a patient receiving chemotherapy is assayed by monitoring the death of normal cells. For example, compositions that enhance hematopoiesis (e.g., increase the number of hematopoietic cells in a patient sample) are useful in the methods of the invention.

The following examples are provided to illustrate the invention, not to limit it. Those skilled in the art will understand that the specific constructions provided below may be changed in numerous ways, consistent with the above described invention while retaining the critical properties of the compounds or combinations thereof.

### EXAMPLES

### Example 1

### GM-CSF Expression in E. coli

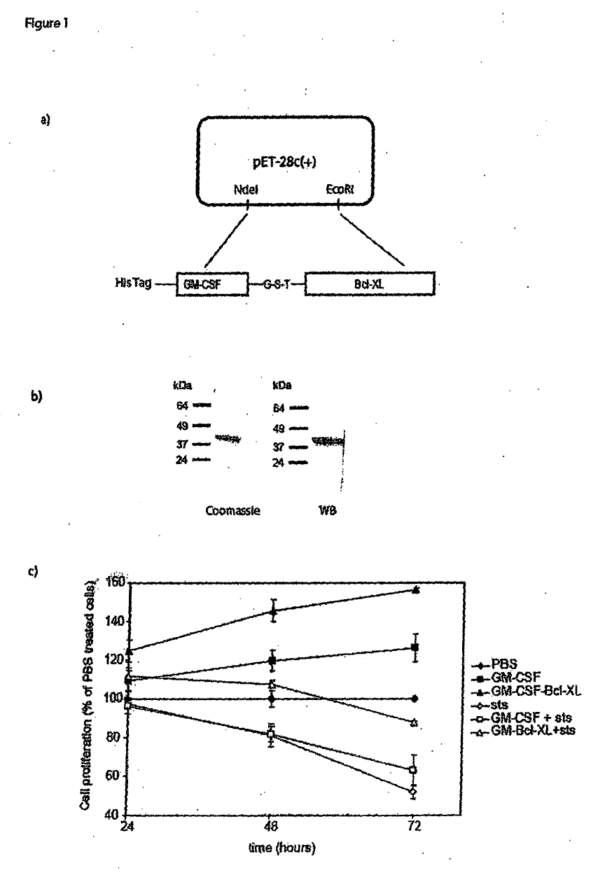

To deliver Bcl-XL into cells of the myeloid lineage, the cDNA for human Bcl-XL was fused to the C-terminus of the gene for human granulocyte-macrophage colony stimulating factor (GM-CSF). A histidine tag is present at the N-terminus of the chimeric protein and this construct was cloned into the expression plasmid pET28b(+) (FIG. 1A). FIG. 1A provides a schematic diagram illustrating the construction of the GM-CSF fusion protein. This construct was cloned into two different expression plasmids. The first plasmid, pET-28a(+) was used for expression in bacteria (E. coli). The second plasmid, pPICZA, was used for expression in the yeast Pichia pastoris. The protein expressed in E. coli was insoluble and found in inclusion bodies. The fusion protein was denatured and, after purification on a His-binding column, the protein was refolded by dilution in the presence of glutathione and arginine. After purification, the protein was ≧90% homogeneous and it had the expected molecular weight, as shown by SDS-PAGE and Western blot (FIG. 1B).

### Example 2

### GM-CSF-Bcl-XL Stimulates HL-60 Proliferation

The GM-CSF-Bcl-XL chimeric protein protected cells from apoptosis more effectively than GM-CSF alone. The effect of GM-CSF-Bcl-XL on the proliferation of a human myeloid cell line, HL-60 was also examined. The GM-CSF-Bcl-XL increased proliferation with the maximum effect observed at 48 hours. At that time the activity was 30% higher than that measured in cells treated with the same molar amount of the cytokine GM-CSF (FIG. 1C).

Staurosporine is a broad specificity inhibitor of various kinases that rapidly induces apoptosis. GM-CSF-Bcl-XL extended HL-60 cell survival in the presence of staurosporine from twenty-four hours to at least seventy-two hours. As shown in FIG. 1C, at forty-eight hours cultures treated with GM-CSF-BclXL and staurosporine contained approximately the same number of cells as control cultures without staurosporine. After seventy-two hours of incubation, 50% of control cells had undergone cell death, while only 20% of cells treated with GM-CSF-BclXL and staurosporine had died. This represents a 30% reduction in cell death resulting from GM-CSF-Bcl-XL treatment. In contrast GM-CSF is not able to block the cytotoxic effect of staurosporine. GM-CSF-Bcl-XL decreases staurosporine cytotoxic activity for at least seventy-two hours.

The GM-CSF-Bcl-XL chimeric protein having a deletion in the Bcl-XL C terminus (GM-CSF-Bcl-XLΔC) was just as effective as the chimeric protein fused to full length Bcl-XL full length. This indicates that the C terminus of Bcl-XL is not essential for the chimeric proteins prosurvival activity. The yield of GM-CSF-Bcl-XL chimeric protein was higher than the yield of GM-CSF-Bcl-XLΔC.

### Example 3

### GM-CSF-Bcl-XL Protected Cells from Tyr-Ag490-Induced Apoptosis

To assess the importance of the Bcl-XL portion of the fusion protein in the chimeric protein's prosurvival activity, the activity of the GM-CSF moiety was inhibited using the kinase inhibitors staurosporine and AgTyr490. Staurosporine was first described as an inhibitor of protein C kinase, but it has recently become clear that staurosporine is a broad specificity inhibitor of a diverse array of different kinases. High affinity binding of GM-CSF to its receptor induces activation of the receptor-associated Jak2 kinase by means of transphosphorylation of the kinase after oligomerization of the receptor subunits. Tyrphostin AG490 (AG490) specifically inhibits the activation of Jak2 blocking leukemic cell growth in vitro and in vivo (Meydan et al., (1996) Nature 379, 645-8; Quelle et al., (1994) Mol Cell Biol 14, 4335-41). Peripheral blood mononuclear cell (PBMC) were incubated with different concentrations of GM-CSF-Bcl-XL in the presence of these two inhibitors for forty-eight hours.

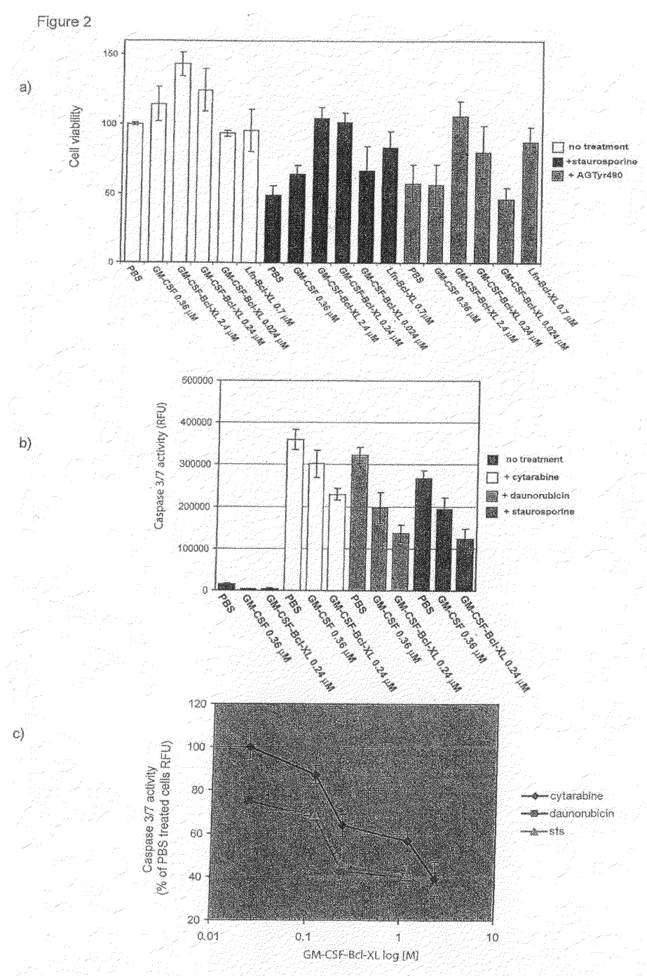

As shown in FIG. 2A, the prosurvival activity of excess GM-CSF was largely inhibited by staurosporine. The prosurvival activity of GM-CSF alone was completely inhibited by AG 490. In contrast, GM-CSF-Bcl-XL protected PBMC from both kinase inhibitors in a dose dependent manner. At 0.24 μM GM-CSF-Bcl-XL, comparable to the molar concentration of GM-CSF used in this experiment, a 50% and 30% increase in cell viability was measured in the presence of staurosporine and AG409 respectively. Thus, Bcl-XL fused with GM-CSF or with the receptor binding domain of the Lethal Factor of Anthrax toxin (Lfn-Bcl-XL) inhibited PBMC apoptosis. GM-CSF-Bcl-XL protected cells from apoptosis even when the prosurvival pathway activated by the GM-CSF portion of the chimeric polypeptide was inhibited.

To determine whether the prosurvival activity of GM-CSF is due to the inhibition of apoptosis, the effect of GM-CSF and GM-CSF-Bcl-XL on cell viability was examined in cells treated with Cytarabine/AraC and daunorubicin. Cytarabine/AraC and daunorubicin apoptosis inducers have been used for the treatment of leukemias and solid tumors (Bruserud et al., (2000) Stem Cells 18, 343-51; Guchelaar et al., (1998) Cancer Chemother Pharmacol 42, 77-83; Guthridge et al., (1998) Stem Cells 16, 301-13; Masquelier et al., (2004) Biochem Pharmacol 67, 1047-56). Caspase 3/7 activity was used as a measure of apoptosis (FIGS. 2B and 2C). Monocytes were treated with cytarabine/AraC or daunorubicin in the presence or the absence of GM-CSF-Bcl-XL. GM-CSF-Bcl-XL was able to reduce the caspase 3/7 apoptotic activity of monocytes treated either Cytarabine/AraC or daunorubicin. GM-CSF-Bcl-XL was more effective in inhibiting caspase 3/7 activity than GM-CSF cytokine alone when each was used at the same concentration (FIG. 2B). The decrease in the catalytic activity of caspase 3/7 was dose-dependent and a concentration of GM-CSF-Bcl-XL of 2.4 μM reduced caspase activity by more than 50% percent.

This indicates that GM-CSF-Bcl-XL inhibited apoptosis thereby increasing cell viability in cells treated with cytotoxic agents. GM-CSF-Bcl-XL combines two activities, the GM-CSF kinase activity and the Bcl-XL apoptosis inhibition to offer a unique approach for myeloprotection.

### Example 4

### GM-CSF-Bcl-XL and GM-CSF-Bcl-XL Mutants Inhibited Apoptosis

To compare the expression, efficacy and importance of the C-terminal amino acids (210-37) of Bcl-XL in mediating the antiapoptotic effect, different constructs were produced. These constructs carried the Bcl-XL at the N-terminal or at the C-terminal of GM-CSF or contained mutations in the C-terminal of Bcl-XL. These constructs were expressed in E. coli. To compare expression, efficacy and the importance of the C-terminal membrane anchor of Bcl-XL, a construct, carrying Bcl-XL (1-209), having a 28 amino acid deletion in the C-terminus (amino acids 210-37) was fused to the C-terminus of GM-CSF. This protein was also expressed in E. coli.

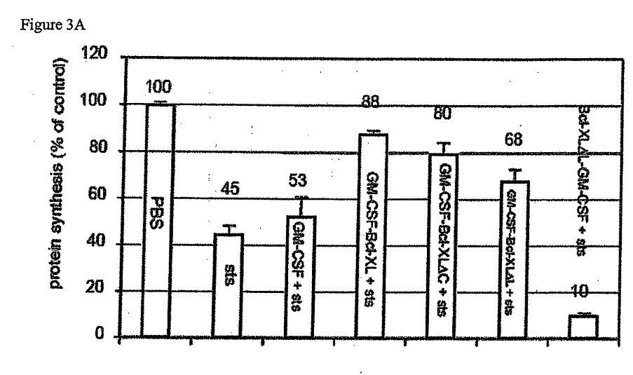

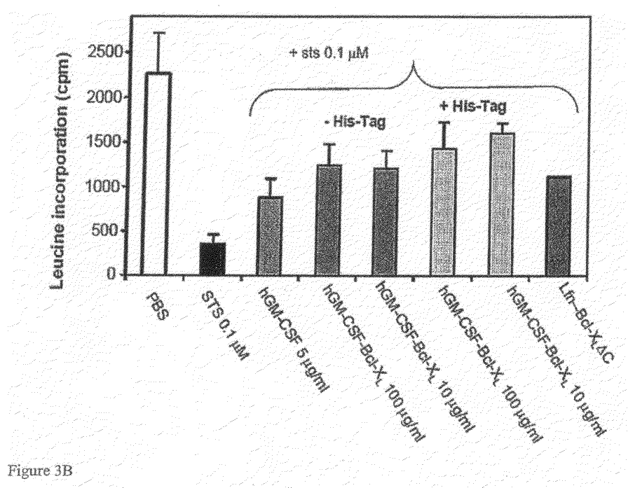

In FIGS. 3A and 3B, the prosurvival effect of the following purified proteins are shown GM-CSF-Bcl-XL and the chimeric mutants GM-CSF-Bcl-XLΔC, GM-CSF-Bcl-XLΔL, and Bcl-XLΔL-GM-CSF. GM-CSF-Bcl-XLDL and Bcl-XLDL-GM-CSF have a deletion of Leu380 (in the chimera). GM-CSF-Bcl-XL-ΔC has the deletion of the segment FNRWFLTGMTVAGVVLLGSLFSRK. The anti-apoptotic activity of the chimera with the Bcl-XL full length C-terminus was comparable to the activity of Bcl-XL containing the deleted C-terminus (amino acids 210-37) (ΔC) (FIG. 3B).

### Example 5

### GM-CSF-Bcl-XL and CD34⁺ Cells

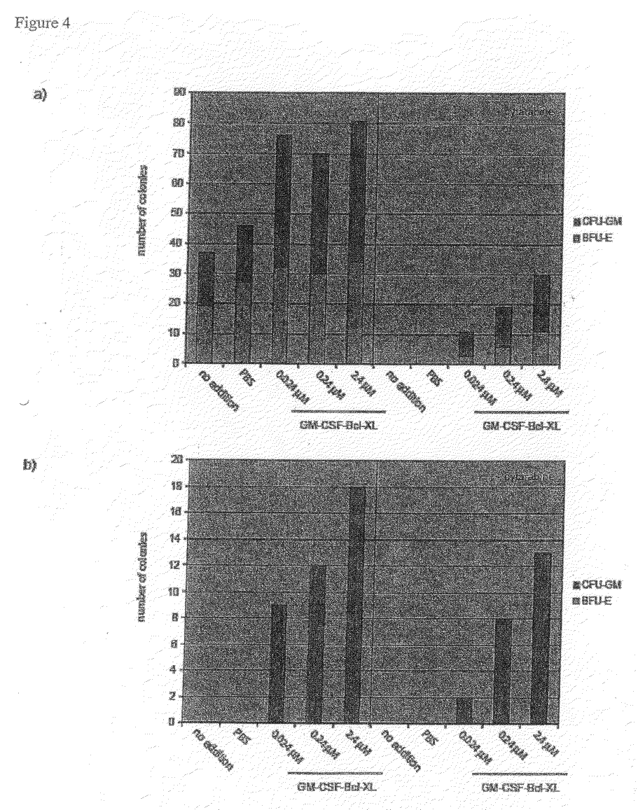

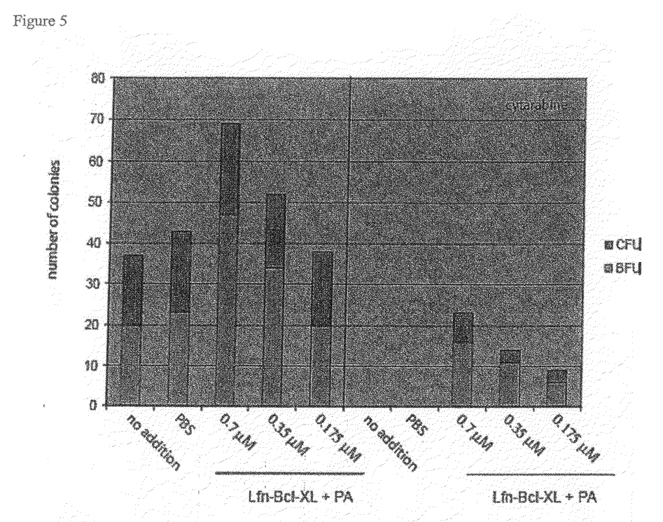

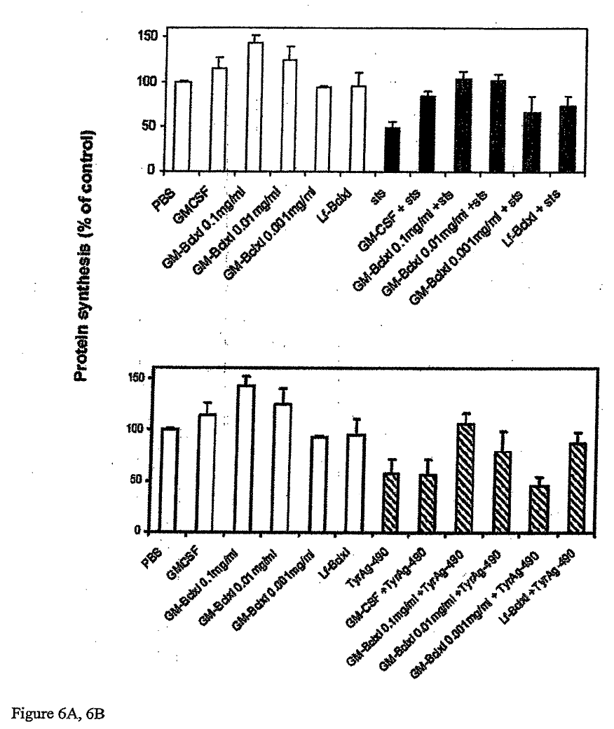

The effect of GM-CSF-Bcl-XL on hematopoiesis was examined using CD34⁺ cell colony assays. The cells were maintained in methylcellulose semisolid medium. CD34⁺ cells isolated from bone marrow were plated in medium supplemented with stem cell factor (SCF), erythropoietin and cytokines. Addition of GM-CSF-Bcl-XL to the culture increased the total number of colonies by two-fold (FIG. 4A). The growth of committed granulocyte-monocyte progenitors (CFU-GM) and burst forming unit-erythroid (BFU-E) colonies was drastically impaired by cytarabine. Incubation of the CD34⁺ cells with GM-CSF-Bcl-XL selectively protected the CFU-GM colonies relative to BFU-E (FIG. 4A). Deprivation of cytokines caused a complete loss of colonies (FIG. 4B). GM-CSF-Bcl-XL protected myeloid precursors from cytokine deprivation, even where the total number of colonies was reduced. The activity of GM-CSF-Bcl-XL protected cells from the effects of cytokine deprivation as well as from the cytotoxic effect of cytarabine, and stimulated the differentiation of precursor cells of the monocyte/macrophage lineage.

CD34⁺ cells cultured in the presence of Lfn-Bcl-XL, containing only Bcl-XL as a prosurvival factor, protected the cells from the cytotoxic effect of cytarabine but the chimera is unable to induce growth or differentiation in essential medium. When cells were deprived of growth factor/cytokines, no colonies were found in wells containing Lfn-Bcl-XL (FIG. 5). In supplemented medium, the Bcl-XL part of the fusion protein increased the number of colonies without any significant difference in differentiated cell type compared to control (cells incubated with or without PBS).

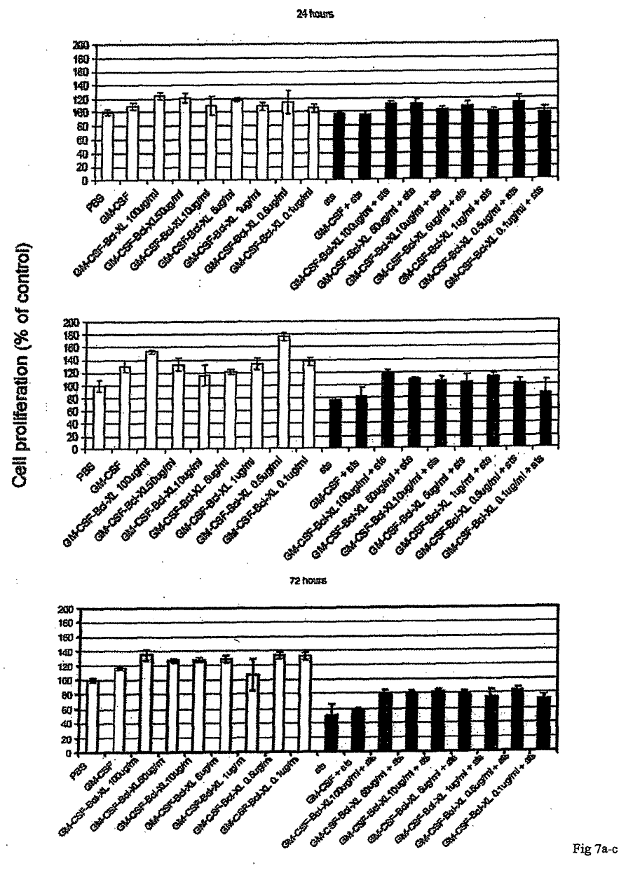

In FIG. 6A macrophage/monocytes purified by adhesion monocyte aphaeresis were treated with human GM-CSF 5 μg/ml; 0.1 mg/ml GM-CSF-Bcl-XL; 0.01 mg/ml GM-CSF-Bcl-XL; or 0.001 mg/ml GM-CSF-Bcl-XL; and a chimeric protein containing the protective antigen binding domain of the anthrax lethal factor (LF) and human Bcl-XL (30 μg/ml) plus the anthrax protective antigen (28 μg/ml) in the presence (black and gray bars) or the absence of staurosporine (0.1 μM) (white bars). In FIG. 6B purified macrophage/monocytes were treated with the following in the absence (white bars) or the presence (striped bars) of the Jak2 kinase inhibitor TyrAg-490 (0.5 μM), for seventy-two hours. The cells were pulsed with ¹⁴C-leucine for 1 hour and harvested. The leucine incorporation was measured and presented as a percentage of the PBS-treated control cells. The mean value was determined from triplicate measurements and are plotted versus the concentration of fusion proteins.

GM-CSF-Bcl-XL binds the GM-CSF receptor and translocates into cells where Bcl-XL blocks cell death.

### Example 6

### Time Course of GM-CSF-Bcl-XL Anti-Apoptotic Activity

In FIGS. 7A, 7B, and 7C, the time course of the effect of GM-CSF-Bcl-XL in the presence of staurosporine is shown. The GM-CSF-Bcl-XL protein protected cells from staurosporine induced apoptosis from twenty-four hours until at least seventy-two hours after induction of apoptosis.

### Example 7

### GM-CSF Expression in Pichia pastoris

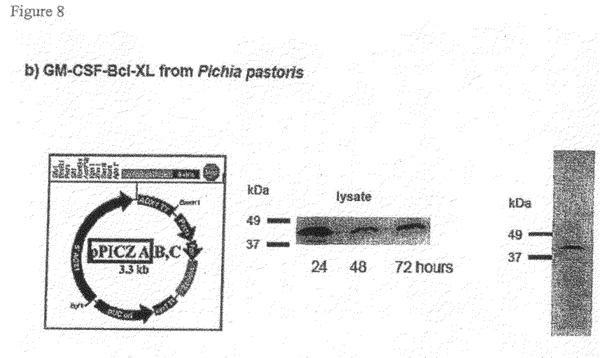

In Pichia, GM-CSF-Bcl-XL was expressed intracellularly. The expression was monitored by Western blot. Production of the chimera was observed at twenty-four hours (FIG. 8). Although high concentrations of proteases inhibitors were used, GM-CSf-Bcl-XL was very sensitive to proteolysis, and the use of protease inhibitors was not always sufficient to eliminate degradation completely. The sensitivity of the GM-CSF-Bcl-XL chimeric polypeptides to proteases can be overcome by the selection of protease resistant variants that retain the cell survival enhancing activity of a chimeric polypeptide of the invention. Methods for the selection of such polypeptides are known in the art and are described herein.

### Example 8

### Pichia and E. coli Produced GM-CSF-Bcl-XL had Anti-Apoptotic Activity

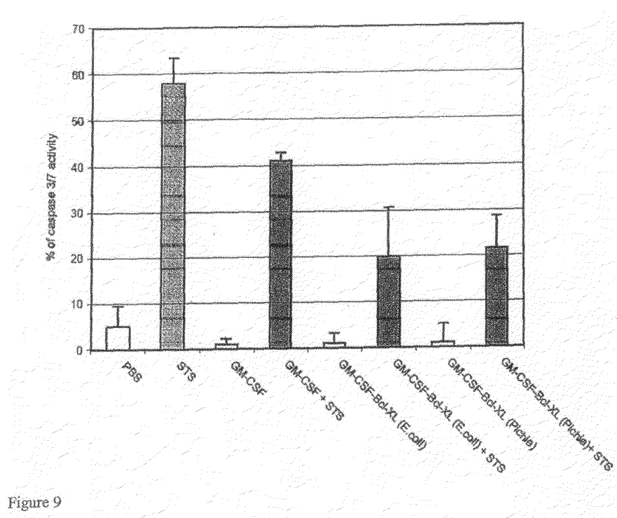

The amount of purified protein was sufficient to confirm that the antiapoptotic effect of Pichia produced GM-CSF-Bcl-XL was comparable to the activity of GM-CSF-Bcl-XL purified from E. coli (FIG. 9). The anti-apoptotic effect was enhanced when Bcl-XL was fused with GM-CSF to form a GM-CSF-Bcl-XL chimera. As expected, the generic kinase inhibitor staurosporine induced apoptosis at the highest levels. Caspase activity was reduced when the GM-CSF cytokine was administered with staurosporine, but levels of caspase activity were reduced by an additional 20% when GM-CSF-Bcl-XL carrying the deletion in the C-terminus of BclXL (amino acids 210-37) was administered with staurosporine (FIG. 2).

The experiments described above were carried out using the following methods and materials.

### Construction and Expression of the Bcl-XL and GM-CSF Fusion Proteins

The cDNA for human GM-CSF was digested with NdeI and BamHI and was then fused with the cDNA of human Bcl-XL (wild-type or truncated form, lacking the C-terminal membrane anchor), which was digested with BglII and EcoRI. The ligation of the two cDNAs, introduced a glycine, serine and threonine as a linker between the two proteins. The fusion genes were then inserted in the E. coli vector pET28b(+) to introduce a His-tag sequence at the N-terminus of the GM-CSF-Bcl-XL (Bcl-XLΔC) cDNA.

Expression of both proteins in E. coli resulted in the production of fusion proteins present in inclusion bodies. Purified proteins were subjected to SDS-PAGE (4-20%) and visualized by Coomassie brilliant blue staining. The fusion gene GM-CSF-Bcl-XL with the His Tag at N-terminus was cloned in the Pichia pastoris expression vector pPICZ A and a stop codon was inserted after the last codon of Bcl-XL. The level of protein expression was monitored by Western blot analysis using an anti-His-Tag antibody. Purified protein was subjected to SDS-PAGE (4-20%) and visualized by Coomassie brilliant blue staining.

### Bacterial Expression of GM-CSF-Bcl-XL

Escherichia coli BL21 DE3 (strain OneShot® BL21DE3, Invitrogen) was used to express GM-CSF-Bcl-XL. Recombinant bacteria transformed with the expression plasmid pET28+ containing the cDNA encoding GM-CSF-Bcl-XL were grown in 1 L of Super Broth (3.2% Tryptone, 2.0% yeast extract, 0.5% NaCl, pH 7.5, KD Medical, Columbia, Md.) containing 50 μg/ml ampicillin (Sigma Chemical Co., St. Louis, Mo.) in 2-liter flasks at 37° C. Protein expression was induced by addition of 1 mM of IPTG (Sigma) when the OD600 reached 0.8-1 OD. After 3 hours incubation, cells were harvested by centrifugation at 5,000 g, and, after resuspension in binding buffer (5 mM imidazole, 20 mM Tris/Cl pH 7.9, 0.5M NaCl), pellets were lysed using a French press. The inclusion bodies with cellular debris were collected by centrifugation at 5000 g and washed four times with 20 ml of binding buffer.

The supernatant from the final centrifugation was removed and the inclusion bodies were dissolved in 30 ml of binding buffer containing 6M guanidine-HCl (3 ml×100 ml culture volume). After incubation on ice for 1 hour to completely dissolve the protein, the insoluble material was removed by centrifugation at 16,000 g for 30 minutes. The supernatant was filtered through a 0.45 micron membrane prior to performing His-Bind purification.

### His-Binding Chromatography.

2.5 ml of a nickel-charged affinity resin used to purify recombinant proteins containing a polyhistidine (6×His) sequence, PROBOND Resin (Invitrogen) was packed under gravity flow in a column 0.5×5 cm. The resin was washed with 5 volumes of pyrogen- and nuclease-free ultrapure water and 5 volumes of binding buffer, containing 6M guanidine-HCl. The column was loaded with the prepared extract and washed with 5 volumes of binding buffer containing 6M guanidine and 10 volumes of washing buffer (60 mM imidazole, 20 mM Tris-Cl pH 7.9, 0.5 M NaCl) containing 6M guanidine-HCl. The bound protein was eluted with 4 volumes of elute buffer (1M imidazole, 20 mM Tris-Cl pH 7.9, 0.5 M NaCl) containing 6M guanidine-HCl. The flow rate during the chromatography was 0.5 ml/min.

### Denaturation and Refolding of GM-CSF-Bcl-XL

The eluted protein was totally denaturated by adding 25 mM DTT to the protein fractions eluted in the 6M guanidine buffer and refolded by dropwise dilution in a 100-fold volume of the refolding buffer (0.1M Tris/Cl pH 8, 0.5M arginine, 1 mM oxidized glutathione) followed by incubation at 25° C. for forty-eight-seventy-two hours. The protein was concentrated in a centrifugal filter device, an Amicon Ultra 15 MWCO 10000 (Millipore, Bedford Mass.), until a concentration≧1 mg/ml and dialyzed against PBS. The quality of purified proteins was analyzed by 4-20% SDS-PAGE stained with Brilliant Blue R, and Western blotting using a His-Tag primary antibody (Novagen, Madison Mass.).

The concentration of GM-CSF-Bcl-XL was determined by a colorimetric assay (BCA kit, Pierce). The final yield of GM-CSF-Bcl-XL was between 2-5 mg/liter of culture. The protein was sterilized by filtration through a 0.22 micron membrane and was stored at 4° C.

### Protein Expression in Pichia Pastoris

cDNA encoding GM-CSF-Bcl-XL was inserted in the EcoRI site in the Pichia intracellular expression vector pPICZ A (Invitrogen) with the His Tag at N-terminus, under the control of the AOX1 promoter. A stop codon was inserted after the last codon of Bcl-XL. The Pichia strain X-33 was transformed by electroporation with the linearized plasmid and transformants were plated on YPDS (Yeast/Peptone/Dextrose/Sorbital) plates containing 100 μg/ml zeocin to isolate the recombinant clones.

Pichia recombinant cells, previous characterized for the expression of GM-CSF-BclXL, were grown in 5 ml of BMGY (1% yeast extract, 2% peptone, 100 mM potassium phosphate, pH 6.0, 1.34% Yeast Nitrogen Base with ammonium sulfate without amino acids, 4×10-5% biotin, 1% glycerol) in 14 ml Falcon round bottomed tube (Becton Dickson Labware) overnight at 30° C. in a shaking incubator (250 rpm). The cells were harvested by centrifuging at 3000 g for 5 minutes and the pellet was resuspended to an OD of 1 in 200 ml BMMY medium (1% yeast extract, 2% peptone, 100 mM potassium phosphate, pH 6.0, 1.34% Yeast Nitrogen Base with ammonium sulfate without amino acids, 4×10-5% biotin, 0.5% methanol) in a 2 L baffled flask to induce expression. The culture was incubated at 30° C. with vigorous shaking (300 rpm) for forty-eight-seventy-two hours. 100% methanol was added every twenty-four hours to a final concentration of 0.5%. Every twenty-four hours, 1 ml of the expression culture was used to analyze expression level and determine the optimal time post-induction to harvest.

The cells were harvested by centrifugation at 5,000 g, and, washed with binding buffer (5 mM imidazole, 20 mM Tris/Cl pH 7.9, 0.5M NaCl) containing 2 protease inhibitor tablets, COMPLETE PROTEASE INHIBITOR COCKTAIL EDTA-free (Roche Diagnostics, Indianapolis, Ind.),/50 ml of buffer. Cells were lysed by adding 100 g of acid washed glass beads (0.5 g of beads/ml of initial culture) with 1 cycle of 5 minutes, frequency 30 Hz, in a mixer mill (Retsch MM200, Haan, Del.). The cellular debris were eliminated by centrifugation at 18,000 g, 5 minutes at 4° C. The supernatant was filtered through a 0.45 micron membrane prior to performing His-Bind purification.

### Protein Purification

The chromatography was performed under the same conditions as the purification of GM-Bcl-XL from E. coli with the same modifications. All buffers used were without guanidine and contained two tablets of COMPLETE PROTEASE INHIBITOR COCKTAIL EDTA free/50 ml of buffer. The fractions were pooled and dialyzed against PBS at 4° C. The concentration of GM-CSF-Bcl-XL was determined by a colorimetric assay (BCA kit, Pierce). Final yield of GM-CSF-Bcl-XL was ˜5 mg/L of culture. The protein was then sterilized by filtration through a 0.22 micron membrane and was stored at 4° C.

### Cell Lines and Cell Viability Assay

The HL-60 cell line, was purchased from the American Type Culture Collection (ATCC). Monocyte aphaeresis was obtained from the NIH Blood Bank. To access the effect of the recombinant proteins, two kinds of assay were performed: cellular protein synthesis inhibition and cell proliferation.

Monocytes from Aphaeresis

Buffy coats and monocytes from aphaeresis of normal healthy donors were obtained from the NIH Blood Bank. PBMC were isolated on Ficoll gradients. The mononuclear cells are resuspended RPMI, 10% FCS (Biofluids, Rockville Md.) and incubated for two hours in tissue culture dishes 150×25 mm. The medium which contains non adherent cells was removed and the cells were washed two times with complete RPMI. The adherent monocytes/macrophages were gently scraped and centrifuged. To access the effect of the recombinant proteins, two kinds of assay were performed: cell proliferation and caspase 3/7 activity. Monocyte/macrophage cells were incubated at concentrations of 1×10⁵ cells/ml in 96-well microtiter plates, overnight, and treated with various concentrations of purified proteins for the required time in Iscove medium, 20% FCS, 10 ng/ml IL3, 10 ng/ml IL6, 10 ng/ml G-CSF. Cell viability was determined with the Celltiter 96 Aqueous One Solution Cell Proliferation Assay kit (Promega, Madison Wis.). The number of viable cells was determined by quantitation of the ATP present, which signals the presence of metabolically active cells. Values given represent the mean of triplicate samples with standard deviation of the mean. Calculation of apoptotic cells was performed using the ApoOne Homogeneous Caspase 3/7 Assay kit (Promega). The caspase 3/7 protease activity was measured as fluorescent intensity subsequent to the cleavage of the substrate Z-DEVD-Rhodamine 110.

### Cellular Protein Synthesis Inhibition

Cellular protein synthesis inhibition was determined as follows. Cells in 100 μl culture media were incubated at concentrations of 1×105 cells/ml in 96-well microtiter plates overnight and treated with various concentrations of purified proteins for the required time in leucine-free RPMI 1640 followed by a 1 hour pulse with 0.1 mCi [¹⁴C]-leucine. Then cells were harvested on glass fiber filters using a commercially available automated cell harvester, PHD cell harvester, (Cambridge Technology, Watertown, Mass.). Radioactivity was counted by liquid scintillation counting. The results were expressed as a percentage of radiolabeled leucine incorporation by PBS-treated control cells.

Cell viability was determined using a colorimetric method for determining the number of viable cells, the Celltiter 96 Aqueous One Solution Cell Proliferation Assay kit (Promega, Madison Wis.). Values given represent the mean of triplicate samples with <10% standard error of the mean. Caspase 3/7 protease activity was measured using the ApoOne Homogeneous Caspase 3/7 Assay kit (Promega).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. An isolated chimeric polypeptide comprising a GM-CSF receptor ligand or polypeptide and a Bcl-xL polypeptide, wherein the chimeric polypeptide specifically binds a GM-CSF receptor and enhances cell survival.

2-7. (canceled)

8. The isolated chimeric polypeptide of claim 1, wherein the chimeric polypeptide inhibits cell death.

9-12. (canceled)

13. The chimeric polypeptide of claim 1, wherein the polypeptide comprises at least a fragment of Bcl-xL capable of inhibiting cell death.

14-16. (canceled)

17. The chimeric polypeptide of claim 1, wherein the polypeptide comprises a fragment selected from the group consisting of
i.
(SEQ ID NO: 19)
APARSPSPSTQPWEHVNAIQEARRLLNLSRDTAAEMNETVEVISEMFD
LQEPTCLQTRLELYKQGLRGSLTKLKGPLTMMASHYKQHCPPTPETSC
ATQTITFESFKENLKDFLLVIPFDCWEPVQE;
ii.
(SEQ ID NO: 20)
EARRLLNLSRD;
and
iii.
(SEQ ID NO: 4)
TMMASHYKQHCPPTPET.

18. The chimeric polypeptide of claim 1, wherein the polypeptide consists essentially of an active fragment of GM-CSF selected from the group consisting of:
i.
(SEQ ID NO: 19)
APARSPSPSTQPWEHVNAIQEARRLLNLSRDTAAEMNETVEVISEMFD
LQEPTCLQTRLELYKQGLRGSLTKLKGPLTMMASHYKQHCPPTPETSC
ATQTITFESFKENLKDFLLVIPFDCWEPVQE;
ii.
(SEQ ID NO: 20)
EARRLLNLSRD;
and
iii.
(SEQ ID NO: 4)
TMMASHYKQHCPPTPET.

19-30. (canceled)

31. An isolated nucleic acid molecule that encodes a chimeric polypeptide of claim 1.

32-37. (canceled)

37. The isolated nucleic acid molecule of claim 36, comprising a nucleic acid molecule having substantial nucleic acid sequence identity to SEQ ID NO: 10.

38-40. (canceled)

41. An isolated polynucleotide capable of encoding a polypeptide having substantial sequence identity to SEQ ID NO: 1, wherein the polypeptide enhances cell survival, promotes cell proliferation, or inhibits cell death.

42. A vector comprising a nucleic acid molecule that encodes a polypeptide of claim 1.

43-47. (canceled)

48. A host cell comprising the vector of claim 42.

49-55. (canceled)

56. A pharmaceutical composition comprising an effective amount of a chimeric polypeptide of claim 1, or fragments thereof, in a pharmaceutically acceptable excipient.

57. A pharmaceutical composition comprising an effective amount of a nucleic acid molecule encoding a chimeric polypeptide of claim 1 in a pharmaceutically acceptable excipient.

58. The pharmaceutical composition of claim 56, wherein the composition further comprises an agent selected from the group consisting of a chemotherapeutic agent, radiation agent, hormonal agent, biological agent, an anti-inflammatory agent, an agent that enhances dopamine production, an anticholinergic, a dopamine mimetic, amantadine, an antithrombotic, and a thrombolytic.

59. A method of enhancing cell survival, the method comprising contacting a cell at risk of cell death with a chimeric polypeptide of claim 1, wherein the contacting enhances cell survival.

60. A method of inhibiting cell death in a cell at risk thereof, the method comprising contacting the cell at risk of cell death with a chimeric polypeptide of claim 1, wherein the contacting inhibits cell death.

61. A method of enhancing cell survival, the method comprising contacting a cell at risk of cell death with a nucleic acid molecule of claim 28, wherein the contacting enhances cell survival.

62. A method of inhibiting cell death in a cell at risk thereof, the method comprising contacting the cell with a nucleic acid molecule of claim 28, wherein the contacting inhibits cell death.

63-69. (canceled)

70. A method of enhancing cell survival in a subject diagnosed as having a disease or disorder characterized by cell death, the method comprising administering to the subject a chimeric polypeptide of claim 1 in an amount effective to enhance cell survival.

71. A method of enhancing cell survival in a subject diagnosed as having a disease or disorder characterized by cell death, the method comprising administering to the subject a nucleic acid molecule encoding the chimeric polypeptide of claim 1 in an amount effective to enhance cell survival.

72-75. (canceled)

76. A method of assessing the efficacy of a cell survival enhancing treatment in a subject, comprising:
determining one or more pre-treatment phenotypes;
administering a therapeutically effective amount of a chimeric polypeptide of claim 1, or a nucleic acid molecule encoding the polypeptide to the subject; and
determining the one or more phenotypes after an initial period of treatment with the an cell death inhibitor; wherein the modulation of the one or more phenotypes indicates efficacy of a cell death inhibitor treatment.

77. A method of selecting a subject having a disease or disorder characterized by cell death for treatment with a cell death inhibitor, comprising:
determining one or more pre-treatment phenotypes,
administering a therapeutically effective amount of a chimeric polypeptide of claim 1, or a nucleic acid molecule encoding the polypeptide to the subject; and
determining the one or more phenotypes after an initial period of treatment with the cell death inhibitor, wherein the modulation of the one or more phenotype is an indication that the disorder is likely to have a favorable clinical response to treatment with a cell death inhibitor.

78-91. (canceled)

92. A method of expanding hematopoietic stem cells or progenitor cells comprising contacting the cells with an effective amount of a polypeptide or nucleic acid molecule of claim 1.


SEQUENCE LISTING (23 sequences)

SEQ ID NO: 1 - protein, 384 aa, Artificial Sequence
  Description of Artificial Sequence Synthetic polypeptide
MGSSHHHHHHSSGLVPRGSHMAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQTITFESFKENLKDFLLVIPFDCWEPVQEGSTMSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 2 - protein, 21 aa, Homo sapiens
MGSSHHHHHHSSGLVPRGSHM

SEQ ID NO: 3 - protein, 10 aa, Homo sapiens
ARRLLNLSRD

SEQ ID NO: 4 - protein, 17 aa, Homo sapiens
TMMASHYKQHCPPTPET

SEQ ID NO: 5 - protein, 4 aa, Homo sapiens
GSTM

SEQ ID NO: 6 - protein, 22 aa, Homo sapiens
SAINGNPSWHLADSPAVNGATG

SEQ ID NO: 7 - protein, 10 aa, Homo sapiens
FELRYRRAFS

SEQ ID NO: 8 - protein, 12 aa, Homo sapiens
GVVLLGSLFSRK

SEQ ID NO: 9 - protein, 126 aa, Homo sapiens
PARSPSPSTQPWEHVNAIQEARRLLNLSRDTAAEMNETVEVISEMFDLQEPTCLQTRLELYKQGLRGSLTKLKGPLTMMASHYKQHCPPTPETSCATQTITFESFKENLKDFLLVIPFDCWEPVQE

SEQ ID NO: 10 - DNA, 1098 bp, Artificial Sequence
  Description of Artificial Sequence Synthetic polynucleotide
catatggcaccagcacgatcgccaagcccaagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggctagccactacaagcagcactgccctccaaccccggaaacttcctgtgcgacccagactatcacctttgaaagtttcaaagagaacctgaaggactttctgcttgtcatcccctttgactgctgggagccagtacaggaaggatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgggcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 11 - DNA, 33 bp, Homo sapiens
gaggcccggcgtctcctgaacctgagtagagac

SEQ ID NO: 12 - DNA, 51 bp, Homo sapiens
accatgatggctagccactacaagcagcactgccctccaaccccggaaact

SEQ ID NO: 13 - DNA, 66 bp, Homo sapiens
agtgccatcaatggcaacccatcctggcacctggcagacagccccgcggtgaatggagccactggg

SEQ ID NO: 14 - DNA, 30 bp, Homo sapiens
tttgaactgcggtaccggcgggcattcagt

SEQ ID NO: 15 - DNA, 39 bp, Homo sapiens
ggcgtggttctgctgggctcactcttcagtcggaaatga

SEQ ID NO: 16 - DNA, 696 bp, Homo sapiens
tctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgggcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaa

SEQ ID NO: 17 - DNA, 5368 bp, Artificial Sequence
  Description of Artificial Sequence Synthetic polynucleotide
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatcccgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 18 - DNA, 3329 bp, Artificial Sequence
  Description of Artificial Sequence Synthetic polynucleotide
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgaggaattcacgtggcccagccggccgtctcggatcggtacctcgagccgcggcggccgccagcttgggcccgaacaaaaactcatctcagaagaggatctgaatagcgccgtcgaccatcatcatcatcatcattgagttttagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 19 - protein, 127 aa, Homo sapiens
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQTITFESFKENLKDFLLVIPFDCWEPVQE

SEQ ID NO: 20 - protein, 11 aa, Homo sapiens
EARRLLNLSRD

SEQ ID NO: 21 - protein, 4 aa, Artificial Sequence
  Description of Artificial Sequence Synthetic peptide
DEVD

SEQ ID NO: 22 - protein, 6 aa, Artificial Sequence
  Description of Artificial Sequence Synthetic 6xHis tag
HHHHHH

SEQ ID NO: 23 - protein, 24 aa, Homo sapiens
FNRWFLTGMTVAGVVLLGSLFSRK